(19) United States
(12) Patent Application Publication
(10) Pub. No.: US 2023/0064189 A1
(43) Pub. Date: Mar. 2, 2023

(54) 1H-PYRAZOLO[3,4-D]PYRIMIDINE COMPOUNDS USEFUL FOR THE TREATMENT OF PLATINUM-RESISTANT CANCER

(71) Applicant: IMPERIAL COLLEGE INNOVATIONS LIMITED, London (GB)

(72) Inventors: Caroline LOW, London (GB); Albert JAXA-CHAMIEC, London (GB); Paula CUNNEA, London (GB); Hani GABRA, London (GB); Evan STRONACH, London (GB); Catherine TRALAU-STEWART, London (GB)

(21) Appl. No.: 17/786,355

(22) PCT Filed: Dec. 18, 2020
(86) PCT No.: PCT/GB2020/053272

§ 371 Date: Jun. 16, 2022

(30) Foreign Application Priority Data
Dec. 19, 2019 (GB) 1918815.0

(51) Int. Cl. A61K 31/519; C07D 487/04; A61K 33/243; A61K 31/555; A61P 35/00

(52) U.S. Cl. CPC A61K 31/519; C07D 487/04; A61K 33/243; A61K 31/555; A61P 35/00

(57) ABSTRACT

The invention relates to compounds of formula (I) and related compounds and their use in the treatment of cancer, especially platinum resistant cancer. The invention also provides a treatment comprising administration of a compound of formula (I) and a platinum containing drug.

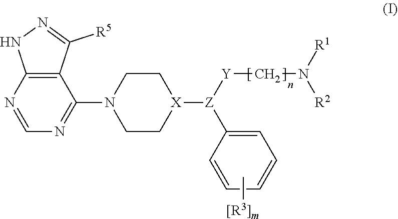

### FIELD OF THE INVENTION

The invention relates to compounds of formula (I) for use in the treatment of cancer, especially platinum resistant cancer. The invention also relates to a treatment comprising administration of a compound of formula (I) and a platinum containing drug. The invention also relates to compounds of formula (II), and their use in the treatment of cancer, especially platinum resistant cancer, as well as a treatment comprising administration of a compound of formula (II) and a platinum containing drug.

### BACKGROUND OF THE INVENTION

It is estimated that over 17 million new cases of cancer occurred worldwide in 2018, and that over 9.6 million cancer deaths occurred in the same year (Cancer Research UK, “Worldwide Cancer”, accessed December 2018). One of the most common treatments of cancers is chemotherapy using a platinum containing drug, such as cisplatin or carboplatin. Cisplatin and carboplatin have been used in the treatment of various cancers, including ovarian cancer, testicular cancer, head and neck cancer, non-small cell lung cancer, sarcomas, lymphomas, bladder cancer, cervical cancer, breast cancer, mesothelioma and pancreatic cancer.

For most relevant cancers, initial response rates to cisplatin or carboplatin are high. However, resistance to those drugs commonly develops after initial treatment. This is known as platinum resistance, and is often defined as tumour progression during or within six months after completion of prior therapy using a platinum containing drug. Many cancers recur as platinum resistant cancers, including lung cancer, breast cancer and ovarian cancer.

Recent studies indicate that platinum resistant tumour cells exist within platinum-sensitive primary tumours at presentation (Schwarz R. F., et al, PLoS Med (2015), Vol 12, e1001789, Cooke S. L., et al, Oncogene (2010), Vol. 29, pages 4905-4913). It is thought that resistant cell selection can occur after initial chemotherapeutic treatment with cisplatin or carboplatin, leading to a platinum resistant tumour.

Treatment of a platinum resistant tumour is more difficult than a platinum sensitive tumour: survival rates for platinum resistant cancers are low. For ovarian cancer, a number of combination treatments have been used for patients with platinum resistant cancer, for example gemcitabine and cisplatin, and paclitaxel and carboplatin. However, due to the response rate for the combination treatments being similar to the mono-therapy for platinum resistant ovarian cancer, no standardized treatment has been developed for these patients.

There remains a need for effective chemotherapeutic treatments for platinum resistant cancers. There is also a need for improved chemotherapeutic treatments for cancers that are treated with platinum containing drugs, for example, improved treatments that can lower the propensity of developing of platinum resistant cancer.

### SUMMARY OF THE INVENTION

The invention provides a compound of formula (I) for use in the treatment of a platinum resistant cancer

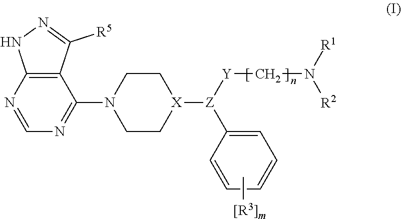

wherein
X is CH, and Z is CR⁴ or N; or X is N, and Z is CR⁴; or X—Z is C═C;
R¹ and R² are independently selected from H, C₁₋₄alkyl, C₃₋₆cycloalkyl and 5-6 membered heterocyclyl, or R¹ and R² together with the nitrogen to which that are attached form a 4-10 membered heterocyclyl ring;
each R³ is independently selected from methyl, halogen and CF₃;
m is 0, 1, 2 or 3;

### R⁴ is H or OH; and

when Z is N, or X—Z is C═C:
Y is selected from bond, —C(O)— and —SO₂NH—, and R⁵ is selected from methyl, halogen and CF₃; or
Y is selected from —SO₂—, and R⁵ is selected from methyl and halogen; or
Y is —C(O)NR⁶—, and R⁵ is halogen;
   when Z is CR⁴:
Y is selected from bond, —C(O)—, —SO₂NH—, and —NHSO₂—, and R⁵ is selected from methyl, halogen and CF₃; or
Y is selected from —SO₂—, and R⁵ is selected from methyl and halogen; or
Y is —C(O)NR⁶—, and R⁵ is halogen;
Y is —NR⁶—, and R⁵ is selected from methyl, halogen and CF₃; or
Y is —O—, and R⁵ is selected from methyl and halogen; and
   when Y is selected from bond, —SO₂NH—, —O—, —NR⁶— and —C(O)NR⁶—, n is 1, 2, 3 or 4;
   when Y is selected from —NHSO₂—, —SO₂— and —C(O)—, n is 0, 1, 2, 3 or 4; and
   R⁶ is selected from H and C₁₋₄alkyl;
   or a salt thereof.

The invention is based on the surprising finding by the inventors that the compounds of formula (I) are particularly effective at restoring platinum sensitivity in platinum resistant cancer cell lines in vitro.

The invention also provides a compound of formula (I) and a platinum containing drug for use in a combined treatment of cancer, whereby the compound of the invention and the platinum containing drug are administered simultaneously or separately. The combination treatment also has particular benefit in the treatment of a cancer that is platinum sensitive but is at risk of developing platinum resistance. By use of the combination, the emergence of platinum resistance can be slowed down, or eliminated.

The invention thus further provides a compound of formula (I) and a platinum containing drug, for use in the treatment of a cancer.

The invention further provides a method for the treatment of platinum containing drug resistant cancer, comprising administering a compound of formula (I) to a subject in need thereof. The invention further provides a method for the treatment of a cancer that is platinum sensitive but which is at risk of developing platinum resistance, comprising administering a compound of formula (I) to a subject in need thereof, and a method for the treatment of a cancer, comprising administering a compound of formula (I) and a platinum containing drug to a subject in need thereof in a combined treatment.

The invention further provides the use of a compound of formula (I) for the manufacture of a medicament for the treatment of a platinum resistant cancer. The invention further provides the use of a compound of formula (I), in combination with a platinum containing drug, for the manufacture of a medicament for the treatment of a cancer that is platinum sensitive but which is at risk of developing platinum resistance. The invention further provides the use of a compound of formula (I), in combination with a platinum containing drug, for the manufacture of a medicament for the treatment of a cancer.

The invention further provides a compound of formula (Ia):

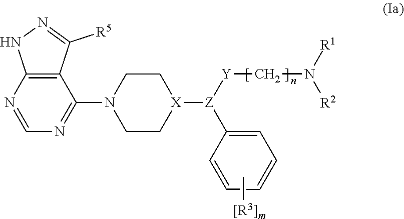

wherein
X is CH, and Z is CR⁴ or N; or X is N, and Z is CR⁴; or X—Z is C═C;
R¹ and R² are independently selected from H, C₁₋₄alkyl, C₃₋₆cycloalkyl and 5-6 membered heterocyclyl, or R¹ and R² together with the nitrogen to which that are attached form a 4-10 membered heterocyclyl ring;
each R³ is independently selected from methyl, halogen and CF₃;
m is 0, 1, 2 or 3;

### R⁴ is H or OH; and

when Z is N, or X—Z is C═C:
Y is selected from bond, —C(O)— and —SO₂NH—, and R⁵ is selected from methyl, halogen and CF₃; or
Y is selected from —SO₂—, and R⁵ is selected from methyl and halogen; or
Y is —C(O)NR⁶—, and R⁵ is halogen;
   when Z is CR⁴:
Y is selected from bond, —C(O)—, —SO₂NH—, and —NHSO₂—, and R⁵ is selected from methyl, halogen and CF₃; or
Y is selected from —SO₂—, and R⁵ is selected from methyl and halogen; or
Y is —C(O)NR⁶—, and R⁵ is halogen;
Y is —NR⁶—, and R⁵ is selected from methyl, halogen and CF₃; or
Y is —O—, and R⁵ is selected from methyl and halogen; and
   when Y is selected from bond, —SO₂NH—, —O—, —NR⁶— and —C(O)NR⁶—, n is 1, 2, 3 or 4;
   when Y is selected from —NHSO₂—, —SO₂— and —C(O)—, n is 0, 1, 2, 3 or 4; and
   R⁶ is selected from H and C₁₋₄alkyl;
   or a salt thereof, with the proviso that compound is not:
2-{[[1-(3-bromo-1H-pyrazolo[3,4-d]pyrimidin-4-yl)piperidin-4-yl](4-chlorophenyl)methyl]oxy}-N,N-dimethylethanamine
3-{[[1-(3-bromo-1H-pyrazolo[3,4-d]pyrimidin-4-yl)piperidin-4-yl](4-chlorophenyl)methyl]oxy}-N,N-dimethylpropan-1-amine
N′-[[1-(3-bromo-1H-pyrazolo[3,4-d]pyrimidin-4-yl)piperidin-4-yl](4-chlorophenyl)methyl]-N,N-diethylethane-1,2-diamine
2-[4-(3-bromo-1H-pyrazolo[3,4-d]pyrimidin-4-yl)piperazin-1-yl]-2-(4-chlorophenyl)-N-[2-(dimethylamino)ethyl]acetamide
N-[1-(3-bromo-1H-pyrazolo[3,4-d]pyrimidin-4-yl)piperidin-4-yl]-N-(4-chlorophenyl)-N′,N′-diethylpropane-1,3-diamine
N-[1-(3-bromo-1H-pyrazolo[3,4-d]pyrimidin-4-yl)piperidin-4-yl]-N-(4-chlorophenyl)-N′-[2-(dimethylamino)ethyl]urea
N-[1-(3-bromo-1H-pyrazolo[3,4-d]pyrimidin-4-yl)piperidin-4-yl]-N-(4-chlorophenyl)-N′,N′-diethylethane-1,2-diamine
N-[[1-(3-bromo-1H-pyrazolo[3,4-d]pyrimidin-4-yl)piperidin-4-yl](4-chlorophenyl)methyl]-N′,N′-diethyl-N-methylethane-1,2-diamine
N-[1-(3-bromo-1H-pyrazolo[3,4-d]pyrimidin-4-yl)piperidin-4-yl]-N-(4-chlorophenyl)-N^{˜}3^{˜},N^{˜}3^{˜}-diethyl-beta-alaninamide
2-{[[1-(3-bromo-1H-pyrazolo[3,4-d]pyrimidin-4-yl)piperidin-4-yl](4-fluorophenyl)methyl]oxy}-N,N-dimethylethanamine
N-[1-(3-bromo-1H-pyrazolo[3,4-d]pyrimidin-4-yl)piperidin-4-yl]-N-(4-chlorophenyl)-2-(diethylamino)ethanesulfonamide
2-{[(R)-[1-(3-bromo-1H-pyrazolo[3,4-d]pyrimidin-4-yl)piperidin-4-yl](4-chlorophenyl)methyl]oxy}-N,N-dimethylethanamine
N-(4-bromo-3-fluorophenyl)-N-[1-(3-bromo-1H-pyrazolo[3,4-d]pyrimidin-4-yl)piperidin-4-yl]-N′-[2-(dimethylamino)ethyl]urea
2-{[(S)-[1-(3-bromo-1H-pyrazolo[3,4-d]pyrimidin-4-yl)piperidin-4-yl](4-chlorophenyl)methyl]oxy}-N,N-dimethylethanamine
3-bromo-4-(4-{(R)-(4-chlorophenyl)[(2-pyrrolidin-1-ylethyl)oxy]methyl}piperidin-1-yl)-1H-pyrazolo[3,4-d]pyrimidine
1-[1-(3-bromo-1H-pyrazolo[3,4-d]pyrimidin-4-yl)piperidin-4-yl]-1-(4-chlorophenyl)-4-(dimethylamino)butan-1-ol
2-{[(R)-[1-(3-bromo-1H-pyrazolo[3,4-d]pyrimidin-4-yl)piperidin-4-yl](4-chloro-3-fluorophenyl)methyl]oxy}-N,N-dimethylethanamine
3-bromo-4-(4-{(R)-(4-chlorophenyl)[(2-piperidin-1-ylethyl)oxy]methyl}piperidin-1-yl)-1H-pyrazolo[3,4-d]pyrimidine
4-[1-(3-bromo-1H-pyrazolo[3,4-d]pyrimidin-4-yl)piperidin-4-yl]-4-(4-chlorophenyl)-N,N-dimethylbutan-1-amine
3-bromo-4-(4-{(R)-(4-chlorophenyl)[(2-morpholin-4-ylethyl)oxy]methyl}piperidin-1-yl)-1H-pyrazolo[3,4-d]pyrimidine
3-bromo-4-(4-{(R)-(4-chloro-3-fluorophenyl)[(2-pyrrolidin-1-ylethyl)oxy]methyl}piperidin-1-yl)-1H-pyrazolo[3,4-d]pyrimidine
2-{[(R)-[1-(3-bromo-1H-pyrazolo[3,4-d]pyrimidin-4-yl)piperidin-4-yl](4-chlorophenyl)methyl]oxy}-N,N-diethylethanamine.

The invention further provides a compound of formula (II):

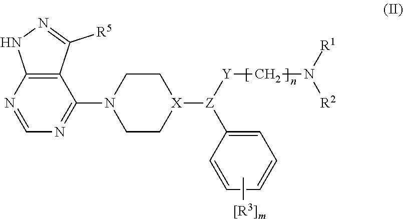

wherein
X is CH, and Z is CR⁴ or N; or X is N, and Z is CR⁴; or X—Z is C═C;
R¹ and R² are independently selected from H, C₁₋₄alkyl, C₃₋₆cycloalkyl and 5-6 membered heterocyclyl, or R¹ and R² together with the nitrogen to which that are attached form a 4 to 10 membered heterocyclyl ring;
each R³ is independently selected from methyl, halogen and CF₃;
m is 0, 1, 2 or 3;

### R⁴ is H or OH;

when Z is N, or X—Z is C═C:
Y is selected from bond, —C(O)— and —SO₂NH—, and R⁵ is selected from methyl, halogen and CF₃; or
Y is —SO₂—, and R⁵ is selected from methyl and halogen; or
Y is —C(O)NR⁶—, and R⁵ is halogen;
   when Z is CR⁴:
Y is selected from bond, —C(O)—, —SO₂NH— and —NHSO₂—, and R⁵ is selected from methyl, halogen and CF₃; or
Y is —SO₂—, and R⁵ is selected from methyl and halogen; or
Y is —C(O)NR⁶—, and R⁵ is halogen;
Y is —NR⁶—, and R⁵ is selected from methyl, halogen and CF₃; or
Y is —O—, and R⁵ is methyl; and
   when Y is selected from —C(O)NR⁶— and —NR⁶—, n is 1, 3 or 4;
   when Y is —SO₂NH— or —O—, n is 1, 2, 3 or 4;
   when Z is CR⁴ or X—Z is C═C, and Y is bond, n is 1, 2 or 4;
   when Z is N, and Y is bond, n is 1 or 4;
   when Z is CR⁴ or X—Z is C═C, and Y is selected from —NHSO₂—, —SO₂— and —C(O)—, n is 0, 1, 2, 3 or 4;
   when Z is N, and Y is selected from —SO₂— and —C(O)—, n is 0, 1, 3 or 4; and
   R⁶ is selected from H and C₁₋₄alkyl;
   or a salt thereof.

### BRIEF DESCRIPTION OF THE DRAWINGS

FIG. 1A shows the isobologram analysis (Loewe synergy and antagonism performed from standard MTT cell viability assay readouts) of concentration ranges (denoted on left side of each isobologram plot) of Example 1 (i), LY3023414 (ii), Afuresertib (iii), Uprosertib (iv), Ipatasertib (v), triciribine (vi), or NU7441 (vii) in combination with concentration ranges (on top side of isobologram plot) of cisplatin in the high grade serous ovarian cancer isogenic paired platinum sensitive cell line PEO1 over 72 hours. Data shown is n=3. Decreasing negative numbers within each table indicate increasing levels of antagonism, increasing positive numbers indicate increasing levels of synergy between cisplatin and inhibitor compounds.

FIG. 1B shows the isobologram analysis (Loewe synergy and antagonism performed from standard MTT cell viability assay readouts) of concentration ranges (denoted on left side of each isobologram plot) of Example 1 (i), LY3023414 (ii), Afuresertib (iii), Uprosertib (iv), Ipatasertib (v), triciribine (vi) or NU7441 (vii) in combination with concentration ranges (on top side of isobologram plot) of cisplatin in the high grade serous ovarian cancer isogenic paired platinum resistant cell line PEO4 over 72 hours. Data shown is n=3. Decreasing negative numbers within each table indicate increasing levels of antagonism, increasing positive numbers indicate increasing levels of synergy between cisplatin and inhibitor compounds.

FIG. 1C shows the isobologram analysis (Loewe synergy and antagonism performed from standard MTT cell viability assay readouts) of concentration ranges (denoted on left side of each isobologram plot) of Example 1 (i), LY3023414 (ii), Afuresertib (iii), Uprosertib (iv), Ipatasertib (v), triciribine (vi) or NU7441 (vii) in combination with concentration ranges (denoted on top side of each isobologram plot) of cisplatin in the platinum resistant epithelial ovarian cancer cell line SKOV3 over 72 hours. Data shown is n=3. Decreasing negative numbers within each table indicate increasing levels of antagonism, increasing positive numbers indicate increasing levels of synergy between cisplatin and inhibitor compounds.

FIG. 2A shows phenotypic apoptosis assay (caspase 3/7 assay readout (Caspase Glo® from Promega) normalised to standard MTT viability assay readout) results following 24 hours incubation in the platinum resistant epithelial ovarian cancer cell line SKOV3 for Example 1 (2 μM), Afuresertib (10 μM), Uprosertib (10 μM) Ipatasertib (20 μM), triciribine (20 μM), NU7441 (10 μM), or LY3023414 (10 μM), plus/minus cisplatin (cddp) at 25 μM. The results are shown as a fold change in apoptosis compared to the vehicle DMSO control, caspase 3/7 data was normalised to cell viability data inferred from corresponding MTT assays (compound concentrations noted on x-axis of graph). Data shown is n=3±s.e.m; ** p<0.01, * p<0.0⁵.

FIG. 2B shows phenotypic apoptosis assay (caspase 3/7 assay readout (Caspase Glo® from Promega) normalised to standard MTT viability assay readout) results following 24 hours incubation in the high grade serous ovarian cancer isogenic paired platinum resistant cell line PEO4 for Example 1 (2 μM), Afuresertib (10 μM), Uprosertib (10 μM), Ipatasertib (20 μM), triciribine (20 μM), NU7441 (10 μM), or LY3023414 (10 μM), plus/minus cisplatin (cddp) at 25 μM. The results are shown as a fold change in apoptosis compared to the vehicle DMSO control, caspase 3/7 data was normalised to cell viability data inferred from corresponding MTT assays (compound concentrations noted on x-axis of graph). Data shown is n=3 s.e.m; ** p<0.01, * p<0.05.

FIG. 3A to 3D show the results of Reverse Phase Protein Array proteomic analysis showing the signalling changes at different response clusters (indicated as response cluster 1 and response cluster 2 on the heatmap of FIG. 3B), or signalling changes induced due to cisplatin or temporal changes, from the results of Reverse Phase Protein Arrays carried out on protein lysates collected following treatment of SKOV3 cells with control (DMSO) or a panel of inhibitor compounds at 6.67 μM plus/minus cisplatin (at 25 μM) (GSK-3g, Roche comp-4, Example 1, Example 2, Example 12a, Example 12b) found to be either inactive or active (increased phosphorylation of AKT at Serine473 and Threonine308 is observed upon treatment with active compounds) at two different time points (2 and 8 hours). FIG. 3A lists the compounds deemed to be active and inactive for the Reverse Phase Protein Array proteomic analysis. FIG. 3B is the heatmap showing the results of the Reverse Phase Protein Array proteomic analysis. FIG. 3C lists proteins decreased following treatment with active compounds (response cluster 1) and proteins increased following treatment with active compounds (response cluster 2). FIG. 3D lists the proteins altered due to cisplatin treatment and temporal effects.

### DETAILED DESCRIPTION

This invention provides a compound of formula (I) for use in the treatment of a platinum resistant cancer.

Inhibition of various kinase pathways have been suggested as potential treatments for cancers. The PI3K-AKT-mTOR pathway and opportunities for interfering with it in treatments of cancer are discussed in several reviews (Janku F., et al, Nat Rev Clin Oncol., 2018, 15, 273-291; LoRusso P. M., J Clin Oncol., 2016, 34, 3802-3815; Fruman D. A. and Rommel, C., Nature Rev Drug Disc., 2014, 10, 140-156). It has been shown that triciribine (1,5-Dihydro-5-methyl-1-β-D-ribofuranosyl-1,4,5,6,8-pentaazaacenaphthylen-3-amine) can re-sensitise clinically platinum resistant ovarian cancer cell lines to cisplatin, leading to apoptosis (Stronach, E. A. et al, Neoplasia, 2011, 13, 1069-80). The present inventors have found that not all compounds described as AKT inhibitors can re-sensitise clinically resistant ovarian cancer cells to cisplatin.

WO 2006/071819 (Exelixis, Inc) discloses a family of [1H-pyrazolo[3,4-D]pyrimidin-4-yl]-piperidine or -piperazine compounds described as Serine-Threonine Kinase Modulators (P70S6k, Akt1 and Akt2) for use in kinase-dependent diseases and conditions, for example cancer, immunological disorders, cardiovascular diseases, inflammatory diseases and degenerative diseases.

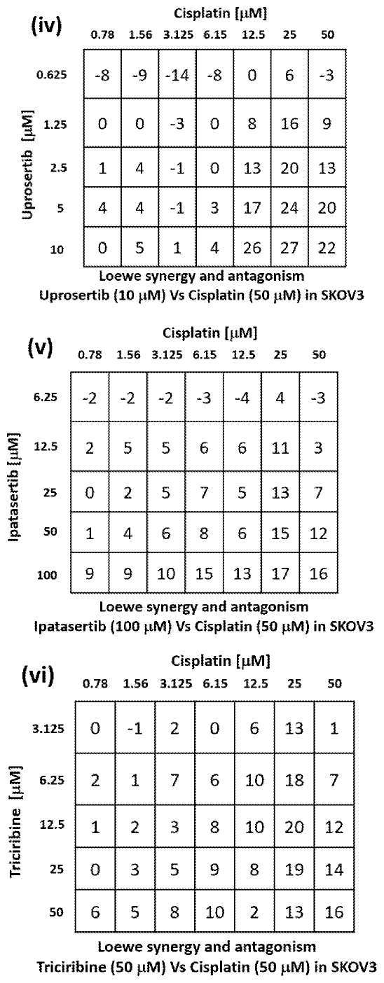

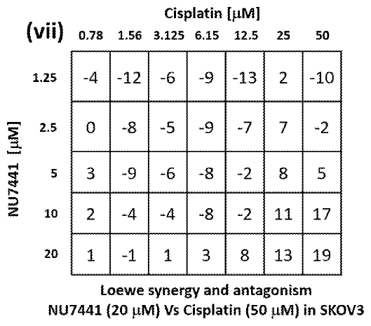

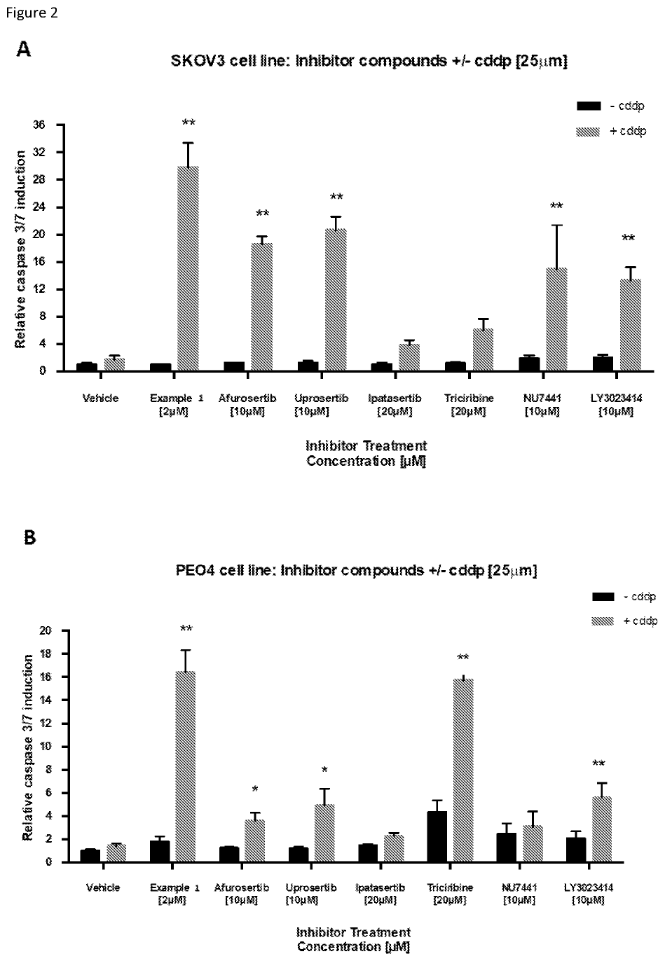

The present inventors have found that compounds of formula (I) are surprisingly effective in the treatment of cancer, in particular platinum resistant cancer. They have found that the combination of a compound of formula (I) of the invention and a platinum containing drug leads to restoration of platinum sensitivity in platinum resistant cell lines in vitro. Compounds of formula (I) have been found to be AKT inhibitors and also have activity as DNA-PK inhibitors, and thus can act as dual AKT/DNA-PK inhibitors. Furthermore, the inventors have found that compounds of the invention are more effective at re-sensitizing platinum resistant cell lines to cisplatin, and at a 5 to 10 fold lower concentration compared to the known AKT inhibitors triciribine, Afuresertib, Uprosertib, and Ipatasertib, the known DNA-PK inhibitor NU7441, and the known mTOR/DNA-PK inhibitor LY3023414 (Samotolisib) as shown in FIGS. 2A and 2B.

For example, when a compound of formula (I) and cisplatin were added to clinically platinum resistant (insensitive) ovarian cancer cells lines, the sensitivity to the cisplatin was restored, leading to increased apoptosis compared with cisplatin alone.

Addition of a compound of formula (I) of the invention and cisplatin to clinically platinum sensitive cell lines can increase apoptosis in the platinum sensitive cells compared with cisplatin alone.

The present invention will have particular utility in the treatment of platinum resistant cancers, including cancers wherein the majority of the tumour cells are platinum resistant, and cancers wherein the tumours contains both platinum sensitive and platinum resistant cells. Examples of platinum resistant cancers include platinum resistant ovarian cancer (for example platinum resistant epithelial ovarian cancer), platinum resistant pancreatic cancer, platinum resistant non-small cell lung cancer, and platinum resistant head and neck cancer.

It will have utility in a patient who is resistant (fully or partially) to treatment using a platinum containing drug.

The present invention will also have utility in cancers that are platinum sensitive, but which are susceptible to recurring in a platinum resistant form, such as ovarian cancer, breast cancer, lung cancer, mesothelioma and pancreatic cancer.

Compounds for use in the present invention, are compounds of formula (I) as set out above (for example compound of formula (Ia), (Ib) or (II)).

The compounds of the invention may be used in the treatment of cancer, for example ovarian cancer, and in particular of platinum resistant cancer, for example platinum resistant ovarian cancer, or cancer that is platinum sensitive but which is at risk of developing platinum resistance, for example ovarian cancer that is platinum sensitive but which is at risk of developing platinum resistance.

In a preferred embodiment, X is CH, and Z is CR⁴ or N; or X is N, and Z is CR⁴.

In a preferred embodiment, R¹ and R² are independently selected from C₁₋₄alkyl and C₃₋₆ cycloalkyl. More preferably, R¹ and R² are each C₁₋₄alkyl, for example the R¹ and R² are each the same C₁₋₄alkyl. Most preferably, R¹ and R² are each methyl.

In one embodiment, R¹ and R² together with the nitrogen to which that are attached form a 4-10 membered heterocyclyl ring. For example, R¹ and R² together with the nitrogen to which that are attached form a 4, 5, 6 or 7 (for example 5 or 7) membered non-aromatic heterocyclyl ring containing 1 nitrogen and optionally 1 or 2 (for example 1) further heteroatoms independently selected from nitrogen, oxygen or sulfur (for example nitrogen or oxygen).

In a preferred embodiment, each R³ is independently selected from methyl, halogen and CF₃ and m is 0, 1 or 2. More preferably R³ is selected from methyl, halogen and CF₃ and m is 0 or 1. More preferably R³ is halogen, for example R³ is selected from fluoro, chloro and bromo, and m is 1. Preferably the R³ group is in the para position. Most preferably R³ is chloro and m is 1, for example m is 1 and R³ is para-chloro.

In a preferred embodiment, R⁴ is H.

In a preferred embodiment, R⁵ is methyl, CF₃ or halogen. More preferably R⁵ is halogen, for example R⁵ is selected from fluoro, chloro and bromo. Most preferably, R⁵ is bromo. In an alternative preferred embodiment R⁵ is methyl.

In a preferred embodiment, Z is CR⁴, and Y is selected from bond, —C(O)— and —NR⁶—, and R⁵ is selected from methyl, halogen and CF₃, or Y is selected from —SO₂— and —O—, and R⁵ is selected from methyl and halogen. More preferably Z is CR⁴, and Y is selected from bond, —C(O)— and —NR⁶—, and R⁵ is selected from methyl and halogen, or Y is selected from —SO₂— and —O—, and R⁵ is selected from methyl and halogen. In such embodiments, when Y is bond, —NR⁶—, —SO₂— or —O—, preferably n is 1, 2, 3 or 4; more preferably 1, 2 or 3; and most preferably 1 or 2. In such embodiments, when Y is —C(O)—, preferably n is 0, 1 or 2; and most preferably 0.

In a preferred embodiment Z is N and Y is selected from bond and —C(O)—, and R⁵ is selected from methyl, halogen and CF₃; or Y is —C(O)NR⁶—, and R⁵ is halogen. More preferably Z is N, and Y is selected from bond and —C(O)—, and R⁵ is selected from methyl and halogen; or Y is —C(O)NR⁶—, and R⁵ is halogen. More preferably Z is N, and Y is bond, and R⁵ is selected from methyl and halogen; or Y is —C(O)NR⁶—, and R⁵ is halogen. In such embodiments, when Y is bond or —C(O)NR⁶—, preferably n is 1, 2, 3 or 4; more preferably 1, 2 or 3; and most preferably 1 or 2. In such embodiments, when Y is —C(O)—, preferably n is 0, 1 or 2; and most preferably 0.

In a preferred embodiment, Z is CR⁴ or N, Y is bond, and R⁵ is halogen, for example bromo, and n is 1 or 2, for example 1; or Y is selected from —SO₂— and —O—, and R⁵ is selected from methyl and halogen, for example methyl and bromo, and n is 1 or 2, for example 2; or Y is —C(O)NR⁶—, and R⁵ is halogen, for example bromo, and n is 1 or 2, for example 2. In such an embodiment, preferably R¹ and R² are each methyl. In such embodiments, when Y is —C(O)NR⁶— or bond, preferably X is N and Z is CR⁴, and R⁴ is H; or when Y is —SO₂—, preferably X is CH and Z is N; or when Y is —O—, preferably X is C, Z is CR⁴, and R⁴ is H. In such embodiments, when Y is —O—, preferably R⁵ is methyl. In such embodiments, when Y is bond, —SO₂— or —C(O)NR⁶— preferably R⁵ is bromo.

In a more preferred embodiment, Z is CR⁴ or N, Y is bond, and R⁵ is halogen, for example bromo, and, n is 1 or 2, for example 1. In such an embodiment, preferably R¹ and R² are each methyl. Alternatively, in such an embodiment preferably R¹ and R² are each methyl and/or preferably X is N, Z is CR⁴, and R⁴ is H.

In a more preferred embodiment, Z is CR⁴ or N, Y is selected from —SO₂— and —O—, and R⁵ is selected from methyl and halogen, for example methyl and bromo, and n is 1 or 2, for example 2. In such an embodiment, preferably R¹ and R² are each methyl. In such embodiments, when Y is —SO₂—, preferably X is CH and Z is N. In such embodiments, when Y is —SO₂—, preferably R⁵ is bromo. In such embodiments, when Y is —O—, preferably X is C, Z is CR⁴, and R⁴ is H. In such embodiments, when Y is —O—, preferably R⁵ is methyl.

In a more preferred embodiment, Z is CR⁴ or N, Y is —C(O)NR⁶—, and R⁵ is halogen, for example bromo, and n is 1 or 2, for example 2. In such an embodiment, preferably R¹ and R² are each methyl. Additionally, or alternatively, in such an embodiment, preferably X is N, Z is CR⁴, and R⁴ is H.

In a more preferred embodiment, Z is CR⁴ or N, Y is —O—, R⁴ is H, and R⁵ is methyl; or Y is bond, R⁴ is H, and R⁵ is halogen, and n is 1 or 2, for example 2. In such an embodiment, preferably X is N.

In a more preferred embodiment, Z is CR⁴ or N, Y is —O—, R⁴ is H, R⁵ is methyl, and n is 1 or 2, for example 2. In such an embodiment, preferably R¹ and R² are each methyl. In such an embodiment, preferably X is N.

In another more preferred embodiment of the invention, Z is CR⁴, and Y is selected from bond, —C(O)—, —O—, —C(O)NR⁶— and —NR⁶—; or Z is N, and Y is —SO₂—. More preferably, Z is CR⁴ and Y is selected from bond, —C(O)—, —O— and —C(O)NR⁶—. More preferably, Z is CR⁴ and Y is bond.

In one very preferred embodiment, Y is bond, R⁴ is H, and R⁵ is halogen, for example bromo, and, n is 1 or 2, for example 1. In such an embodiment, preferably R¹ and R² are each methyl. In such an embodiment, preferably X is N. In such an embodiment, preferably R³ is selected from methyl, halogen and CF₃ and m is 0 or 1, for example R³ is halogen and m is 1. Preferably, the R³ group is in the para position. More preferably, R³ is chloro and m is 1, for example m is 1 and R³ is para-chloro.

In a preferred embodiment, when Y is selected from bond, —O—, —NR⁶—, and —C(O)NR⁶—, n is 1, 2 or 3; or when Y is selected from —SO₂— and —C(O)—, n is 0, 1, 2 or 3. More preferably, when Y is selected from bond, —O—, —NR⁶—, and —C(O)NR⁶—, n is 1 or 2; or when Y is —SO₂—, n is 1 or 2; or when Y is —C(O)—, n is 0.

For the avoidance of doubt, an embodiment or preferred aspect of any one feature of a compound of the invention may be combined with any embodiment or preferred aspect of another feature of a compound of the invention to create a further embodiment.

Examples of compounds of formula (I) include:
2-(4-(3-bromo-1H-pyrazolo[3,4-d]pyrimidin-4-yl)piperazin-1-yl)-2-(4-chlorophenyl)-N,N-dimethylethan-1-amine;
2-((1-(3-bromo-1H-pyrazolo[3,4-d]pyrimidin-4-yl)piperidin-4-yl)(4-chlorophenyl)methoxy)-N,N-dimethylethan-1-amine;
2-(4-(3-bromo-1H-pyrazolo[3,4-d]pyrimidin-4-yl)piperazin-1-yl)-2-(4-chlorophenyl)-N-(2-(dimethylamino)ethyl)acetamide;
(−)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
(+)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
2-(4-(3-bromo-1H-pyrazolo[3,4-d]pyrimidin-4-yl)piperazin-1-yl)-2-(4-chlorophenyl)-N,N-dimethylacetamide;
2-((4-chlorophenyl)(1-(3-methyl-1H-pyrazolo[3,4-d]pyrimidin-4-yl)piperidin-4-yl)methoxy)-N,N-dimethylethan-1-amine;
N-(1-(3-bromo-1H-pyrazolo[3,4-d]pyrimidin-4-yl)piperidin-4-yl)-N-(4-chlorophenyl)-2-(diethylamino)ethane-1-sulfonamide; and
2-(1-(3-bromo-1H-pyrazolo[3,4-d]pyrimidin-4-yl)piperidin-4-yl)-2-(4-chlorophenyl)-N,N-dimethylethan-1-amine;
N¹-((1-(3-bromo-1H-pyrazolo[3,4-d]pyrimidin-4-yl)piperidin-4-yl)(4-chlorophenyl)methyl)-N²,N²-dimethylethane-1,2-diamine;
2-(4-chlorophenyl)-N,N-dimethyl-2-(1-(3-(trifluoromethyl)-1H-pyrazolo[3,4-d]pyrimidin-4-yl)piperidin-4-yl)ethan-1-amine;
N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(+)-N-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(−)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
2-(4-chlorophenyl)-N,N-dimethyl-2-(4-(3-(trifluoromethyl)-1H-pyrazolo[3,4-d]pyrimidin-4-yl)piperazin-1-yl)ethan-1-amine;
1-(1-(3-bromo-1H-pyrazolo[3,4-d]pyrimidin-4-yl)piperidin-4-yl)-1-(4-chlorophenyl)-5-(dimethylamino)pentan-1-ol;
4-(4-chlorophenyl)-N,N-dimethyl-4-(1-(3-(trifluoromethyl)-1H-pyrazolo[3,4-d]pyrimidin-4-yl)piperidin-4-ylidene)butan-1-amine;
1-(1-(3-bromo-1H-pyrazolo[3,4-d]pyrimidin-4-yl)piperidin-4-yl)-1-(4-chlorophenyl)-4-(dimethylamino)butan-1-ol; and
3-((1-(3-bromo-1H-pyrazolo[3,4-d]pyrimidin-4-yl)piperidin-4-yl)(4-chlorophenyl)methoxy)-N,N-dimethylpropan-1-amine.

Preferred compounds of formula (I) are:
2-(4-(3-bromo-1H-pyrazolo[3,4-d]pyrimidin-4-yl)piperazin-1-yl)-2-(4-chlorophenyl)-N,N-dimethylethan-1-amine;
2-((1-(3-bromo-1H-pyrazolo[3,4-d]pyrimidin-4-yl)piperidin-4-yl)(4-chlorophenyl)methoxy)-N,N-dimethylethan-1-amine;
2-(4-(3-bromo-1H-pyrazolo[3,4-d]pyrimidin-4-yl)piperazin-1-yl)-2-(4-chlorophenyl)-N-(2-(dimethylamino)ethyl)acetamide;
(−)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
(+)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
2-(4-(3-bromo-1H-pyrazolo[3,4-d]pyrimidin-4-yl)piperazin-1-yl)-2-(4-chlorophenyl)-N,N-dimethylacetamide;
2-((4-chlorophenyl)(1-(3-methyl-1H-pyrazolo[3,4-d]pyrimidin-4-yl)piperidin-4-yl)methoxy)-N,N-dimethylethan-1-amine;
N-(1-(3-bromo-1H-pyrazolo[3,4-d]pyrimidin-4-yl)piperidin-4-yl)-N-(4-chlorophenyl)-2-(diethylamino)ethane-1-sulfonamide; and
2-(1-(3-bromo-1H-pyrazolo[3,4-d]pyrimidin-4-yl)piperidin-4-yl)-2-(4-chlorophenyl)-N,N-dimethylethan-1-amine.

The present invention also provides compounds of formula (Ia), as set described above, as well as the use of compounds of formula (Ia) in the treatments and methods described herein. In particular, the compounds of formula (Ia) may be used in the treatment of cancer, for example ovarian cancer, and in particular of platinum resistant cancer, for example platinum resistant ovarian cancer. In one embodiment, the ovarian cancer is high-grade serous ovarian cancer, for example platinum resistant high-grade serous ovarian cancer. The preferred embodiments for a compound of formula (I) are equally applicable for a compound of formula (1a).

In certain embodiments, preferred compounds of formula (Ia) are:
2-[4-(3-bromo-1H-pyrazolo[3,4-d]pyrimidin-4-yl)piperazin-1-yl]-2-(4-chlorophenyl)-N,N-dimethyl-ethanamine;
2-(4-(3-bromo-1H-pyrazolo[3,4-d]pyrimidin-4-yl)piperazin-1-yl)-2-(4-chlorophenyl)-N,N-dimethylacetamide;
2-((4-chlorophenyl)(1-(3-methyl-1H-pyrazolo[3,4-d]pyrimidin-4-yl)piperidin-4-yl)methoxy)-N,N-dimethylethan-1-amine
2-(1-(3-bromo-1H-pyrazolo[3,4-d]pyrimidin-4-yl)piperidin-4-yl)-2-(4-chlorophenyl)-N,N-dimethylethan-1-amine;
(−)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
(+)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
N¹-((1-(3-bromo-1H-pyrazolo[3,4-d]pyrimidin-4-yl)piperidin-4-yl)(4-chlorophenyl)methyl)-N²,N²-dimethylethane-1,2-diamine;
2-(4-chlorophenyl)-N,N-dimethyl-2-(1-(3-(trifluoromethyl)-1H-pyrazolo[3,4-d]pyrimidin-4-yl)piperidin-4-yl)ethan-1-amine;
N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(+)-N-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(−)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
2-(4-chlorophenyl)-N,N-dimethyl-2-(4-(3-(trifluoromethyl)-1H-pyrazolo[3,4-d]pyrimidin-4-yl)piperazin-1-yl)ethan-1-amine;
1-(1-(3-bromo-1H-pyrazolo[3,4-d]pyrimidin-4-yl)piperidin-4-yl)-1-(4-chlorophenyl)-5-(dimethylamino)pentan-1-ol; and
4-(4-chlorophenyl)-N,N-dimethyl-4-(1-(3-(trifluoromethyl)-1H-pyrazolo[3,4-d]pyrimidin-4-yl)piperidin-4-ylidene)butan-1-amine.

In certain embodiments, preferred compounds of formula (Ia) are:
2-(4-(3-bromo-1H-pyrazolo[3,4-d]pyrimidin-4-yl)piperazin-1-yl)-2-(4-chlorophenyl)-N,N-dimethylethan-1-amine;
2-((1-(3-bromo-1H-pyrazolo[3,4-d]pyrimidin-4-yl)piperidin-4-yl)(4-chlorophenyl)methoxy)-N,N-dimethylethan-1-amine;
2-(4-(3-bromo-1H-pyrazolo[3,4-d]pyrimidin-4-yl)piperazin-1-yl)-2-(4-chlorophenyl)-N-(2-(dimethylamino)ethyl)acetamide;
2-(4-(3-bromo-1H-pyrazolo[3,4-d]pyrimidin-4-yl)piperazin-1-yl)-2-(4-chlorophenyl)-N,N-dimethylacetamide;
2-((4-chlorophenyl)(1-(3-methyl-1H-pyrazolo[3,4-d]pyrimidin-4-yl)piperidin-4-yl)methoxy)-N,N-dimethylethan-1-amine;
N-(1-(3-bromo-1H-pyrazolo[3,4-d]pyrimidin-4-yl)piperidin-4-yl)-N-(4-chlorophenyl)-2-(diethylamino)ethane-1-sulfonamide; and
2-(1-(3-bromo-1H-pyrazolo[3,4-d]pyrimidin-4-yl)piperidin-4-yl)-2-(4-chlorophenyl)-N,N-dimethylethan-1-amine.

The present invention also provides compounds of formula (II), as described out above as well as the use of compounds of formula (II) in the treatments and methods described herein. In particular, the compounds of formula (II) may be used in the treatment of cancer, for example ovarian cancer, and in particular of platinum resistant cancer, for example platinum resistant ovarian cancer. In one embodiment, the ovarian cancer is high-grade serous ovarian cancer, for example platinum resistant high-grade serous ovarian cancer.

In a preferred embodiment, X is CH, and Z is CR⁴; or X is N, and Z is CR⁴; or X—Z is C═C. More preferably, X is CH, and Z is CR⁴; or X is N, and Z is CR⁴.

In a preferred embodiment, R¹ and R² are independently selected from C₁₋₄alkyl and C₃₋₆cycloalkyl. More preferably R¹ and R² are each C₁₋₄alkyl, and most preferably R¹ and R² are each methyl.

In one embodiment, R¹ and R² together with the nitrogen to which that are attached form a 4-10 membered heterocyclyl ring. For example, R¹ and R² together with the nitrogen to which that are attached form a 4, 5, 6 or 7 (for example 5 or 7) membered non-aromatic heterocyclyl ring containing 1 nitrogen and optionally 1 or 2 (for example 1) further heteroatoms independently selected from nitrogen, oxygen or sulfur (for example nitrogen or oxygen).

In a preferred embodiment, each R³ is independently selected from methyl, halogen and CF₃ and m is 0, 1 or 2. More preferably R³ is selected from methyl, halogen and CF₃ and m is 0 or 1. More preferably R³ is halogen, for example R³ is selected from fluoro, chloro and bromo, and m is 1. Preferably the R³ group is in the para position. Most preferably R³ is chloro and m is 1, for example m is 1 and R³ is para-chloro.

In a preferred embodiment R⁴ is H.

In a preferred embodiment, R⁵ is halogen or methyl. More preferably R⁵ is halogen, for example R⁵ is selected from fluoro, chloro and bromo. Even more preferably, R⁵ is bromo. In alternative more preferred embodiment, R⁵ is methyl.

In a preferred embodiment, Y is selected from bond, —C(O)— and —NR⁶—, and R⁵ is selected from halogen and CF₃, more preferably R⁵ is halogen, for example bromo; or Y is —O—, R⁴ is H or OH, and R⁵ is methyl. In such an embodiment, preferably X is CH, and Z is CR⁴; or X is N, and Z is CR⁴.

In a preferred embodiment when Z is CR⁴, n is 1, 2 or 3, and Y is —O—; or n is 1 or 3 and Y is selected from —NR⁶— and —C(O)NR⁶; or n is 1 or 2 and Y is bond; or n is 0, 1, 2 or 3 and Y is selected from —SO₂— and —C(O)—. More preferably, n is 1 or 2 and Y is selected from bond and —O—, or n is 1 and Y is selected from —NR⁶— and —C(O)NR⁶—; or n is 0 or 1 and Y is —C(O)— or —SO₂—. More preferably when Y is selected from —SO₂— and —C(O)—, n is 0. In such an embodiment, preferably X is CH, and Z is CR⁴; or X is N, and Z is CR⁴.

In one especially preferred embodiment, n is 1 or 2 and Y is bond, for example n is 1 and Y is bond. In such embodiments, preferably X is CH, and Z is CR⁴.

In another preferred embodiment, when Y is —NR⁶—, —SO₂— or —O—, R⁵ is preferably selected from methyl, fluoro, chloro and bromo; more preferably, R⁵ is methyl or bromo; most preferably, R⁵ is bromo. In such an embodiment, preferably X is CH, and Z is CR⁴; or X is N, and Z is CR⁴.

In a preferred embodiment Z is CR⁴, Y is selected from bond, —C(O)— and —NR⁶—, R⁴ is H, and R⁵ is selected from halogen and CF₃, preferably R⁵ is halogen, for example bromo; or Y is —SO₂—, R⁴ is H, and R⁵ is selected from methyl and halogen, preferably R⁵ is halogen, for example bromo; Y is —O—, R⁴ is H, and R⁵ is methyl; or Y is —C(O)NR⁶—, R⁴ is H, and R⁵ is halogen, for example bromo. In such embodiments, preferably, when Y is selected from bond and —O—, n is 1 or 2; or when Y is selected from —NR⁶— and —C(O)NR⁶—, n is 1; or when Y is selected from —SO₂— and —C(O)—, n is 0 or 1. More preferably when Y is selected from —SO₂— and —C(O)—, n is 0.

In one especially preferred embodiment, n is 1 or 2 and Y is bond, for example n is 1 and Y is bond, and R⁵ is halogen, for example bromo. In such embodiments, preferably, X is CH, and Z is CR⁴.

In a more preferred embodiment Z is CR⁴, Y is selected from bond and —C(O)—, R⁴ is H, and R⁵ is selected from halogen and CF₃ preferably R⁵ is halogen, for example bromo; or Y is —O—, R⁴ is H or OH, and R⁵ is methyl. More preferably, Y is bond, R⁴ is H, and R⁵ is selected from halogen and CF₃ preferably R⁵ is halogen, for example bromo; Y is —O—, R⁴ is H or OH, and R⁵ is methyl. Preferably, when Y is selected from bond and —O—, n is 1 or 2; or when Y is —C(O)—, n is 0. In such an embodiment, preferably X is CH, and Z is CR⁴; or X is N, and Z is CR⁴.

In another more preferred embodiment Z is CR⁴, Y is —O—, R⁴ is H, and R⁵ is methyl; or Y is bond, R⁴ is H, R⁵ is halogen, and n is 1 or 2, for example 2. In such an embodiment, preferably R¹ and R² are each methyl.

In another more preferred embodiment Z is CR⁴, Y is —O—, and R⁵ is methyl, and n is 1 or 2, for example 2. In such an embodiment, preferably R¹ and R² are each methyl. In such an embodiment, preferably X is CH.

In one very preferred embodiment, Z is CR⁴, Y is bond, R⁴ is H, and R⁵ is halogen, for example bromo, and, n is 1 or 2, for example 1. In such an embodiment, preferably R¹ and R² are each methyl. In such an embodiment, preferably X is N.

In another preferred embodiment:

X is CH, and Z is CR⁴; or X is N, and Z is CR⁴; or X—Z is C═C;
R¹ and R² are independently selected from H, C₁₋₄alkyl, C₃₋₆cycloalkyl and 5-6 membered heterocyclyl, or R¹ and R² together with the nitrogen to which that are attached form a 4 to 10 membered heterocyclyl ring; more preferably R¹ and R² are each C₁₋₄alkyl, most preferably R¹ and R² are each methyl;
m is 0, 1, 2 or 3, and R³ is independently selected from methyl, halogen and CF₃; more preferably m is 1 and R³ is chloro; most preferably m is 1 and R³ is para-chloro.
R⁴ is H or OH; more preferably R⁴ is H;
R⁶ is selected from H and C₁₋₄alkyl; more preferably R⁶ is H;
Y is selected from bond, —C(O)— and —NR⁶—, and R⁵ is selected from methyl, halogen and CF₃; or
Y is —SO₂—, and R⁵ is selected from methyl and halogen; or
Y is —O—, and R⁵ is methyl; or
Y is —C(O)NR⁶—, and R⁵ is halogen;
more preferably Y is selected from bond, —C(O)—, —O— and —NR⁶—; most preferably Z is CR⁴, Y is —O—, and R⁵ is methyl; and/or Y is selected from bond, —C(O)— and —NR⁶— and R⁵ is bromo;
when Y is —O—, n is 1, 2, 3 or 4; or when Y is —C(O)NR⁶— or —NR⁶—, n is 1, 3 or 4; or when Y is bond, n is 1, 2 or 4; or when Y is selected from —SO₂— and —C(O)—, n is 0, 1, 2, 3 or 4; more preferably when Y is —O—, n is 1, 2 or 3; or when Y is —C(O)NR⁶— or —NR⁶, n is 1 or 3-; or when Y is bond, n is 1 or 2; or when Y is selected from —SO₂— and —C(O)—, n is 0, 1 or 2;
or a salt thereof.

Examples of compounds of formula (II) are:
2-[4-(3-bromo-1H-pyrazolo[3,4-d]pyrimidin-4-yl)piperazin-1-yl]-2-(4-chlorophenyl)-N,N-dimethyl-ethanamine;
2-(4-(3-bromo-1H-pyrazolo[3,4-d]pyrimidin-4-yl)piperazin-1-yl)-2-(4-chlorophenyl)-N,N-dimethylacetamide;
2-((4-chlorophenyl)(1-(3-methyl-1H-pyrazolo[3,4-d]pyrimidin-4-yl)piperidin-4-yl)methoxy)-N,N-dimethylethan-1-amine;
2-(1-(3-bromo-1H-pyrazolo[3,4-d]pyrimidin-4-yl)piperidin-4-yl)-2-(4-chlorophenyl)-N,N-dimethylethan-1-amine;
(−)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
(+)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
N¹-((1-(3-bromo-1H-pyrazolo[3,4-d]pyrimidin-4-yl)piperidin-4-yl)(4-chlorophenyl)methyl)-N²,N²-dimethylethane-1,2-diamine;
2-(4-chlorophenyl)-N,N-dimethyl-2-(1-(3-(trifluoromethyl)-1H-pyrazolo[3,4-d]pyrimidin-4-yl)piperidin-4-yl)ethan-1-amine;
N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(+)-N-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(−)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
2-(4-chlorophenyl)-N,N-dimethyl-2-(4-(3-(trifluoromethyl)-1H-pyrazolo[3,4-d]pyrimidin-4-yl)piperazin-1-yl)ethan-1-amine;
1-(1-(3-bromo-1H-pyrazolo[3,4-d]pyrimidin-4-yl)piperidin-4-yl)-1-(4-chlorophenyl)-5-(dimethylamino)pentan-1-ol;
4-(4-chlorophenyl)-N,N-dimethyl-4-(1-(3-(trifluoromethyl)-1H-pyrazolo[3,4-d]pyrimidin-4-yl)piperidin-4-ylidene)butan-1-amine.

Preferred compounds of formula (II) are:
2-[4-(3-bromo-1H-pyrazolo[3,4-d]pyrimidin-4-yl)piperazin-1-yl]-2-(4-chlorophenyl)-N,N-dimethyl-ethanamine;
N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(+)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine; and
(−)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine.

For the avoidance of doubt, an embodiment or preferred aspect of any one feature of a compound of formula (II) may be combined with any embodiment or preferred aspect of another feature of a compound of formula (II) to create a further embodiment.

For certain uses, the compound may be a compound of formula (Ib)

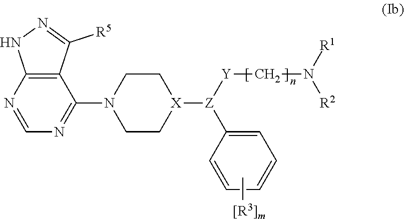

wherein
X is CH, and Z is CR⁴ or N; or X is N, and Z is CR⁴; or X—Z is C═C;
R¹ and R² are independently selected from H, C₁₋₄alkyl, C₃₋₆cycloalkyl and 5-6 membered heterocyclyl, or R¹ and R² together with the nitrogen to which that are attached form a 4-10 membered heterocyclyl ring;
each R³ is independently selected from methyl, halogen and CF₃;
m is 0, 1, 2 or 3;

### R⁴ is H or OH; and

when Z is CR⁴ or N, or X—Z is C═C, Y is selected from bond, —SO₂NH—, —SO₂—, —C(O)NR⁶— and —C(O)—;
when Z is CR⁴, Y is selected from —NHSO₂—, —NR⁶— and —O—; and
when Y is selected from bond, —SO₂NH—, —O—, —NR⁶— and —C(O)NR⁶—, n is 1, 2, 3 or 4;
when Y is selected from —NHSO₂—, —SO₂— and —C(O)—, n is 0, 1, 2, 3 or 4; and
R⁵ is selected from methyl, halogen and CF₃; and
R⁶ is selected from H and C₁₋₄alkyl;
or a salt thereof.

The preferred embodiments for a compound of formula (I) are equally applicable for a compound of formula (Ib).

The present inventors have also found that the combination of a compound of formula (III) and a platinum containing drug leads to restoration of platinum sensitivity in platinum resistant cell lines in vitro. Thus the present invention also provides the use of compounds of formula (III) in the treatments and methods described herein. In particular, the compounds of formula (III) may be used in the treatment of cancer, for example ovarian cancer, and in particular of platinum resistant cancer, for example platinum resistant ovarian cancer. In one embodiment, the ovarian cancer is high-grade serous ovarian cancer, for example platinum resistant high-grade serous ovarian cancer. The present invention also provides a compound of formula (III) in combination with a platinum containing drug, for example for use in the treatment of cancer, in particular platinum resistant cancer. The compound of formula (III) is an AKT inhibitor; it also has activity as a DNK-PK inhibitor. The compound of formula (III) has the following structure.

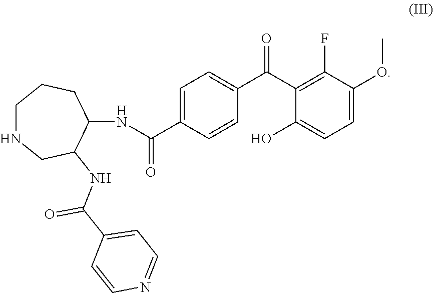 [SMILES: COc1ccc(O)c(C(=O)c2ccc(C(=O)NC3CCCNCC3NC(=O)c3ccncc3)cc2)c1F]

In one preferred embodiment, the compound of formula (III) has the following structure:

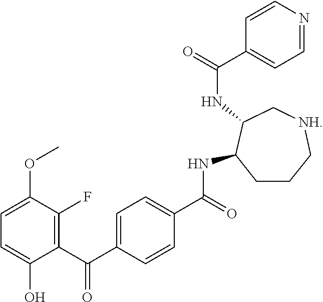 [SMILES: COc1ccc(O)c(C(=O)c2ccc(C(=O)N[C@@H]3CCCNC[C@H]3NC(=O)c3ccncc3)cc2)c1F]

Suitable salts of a compound of the invention (for example a compound of formula (I), (Ia), (Ib), (II) or (III)), or for use in the invention, include those formed with organic or inorganic acids or bases. In particular, suitable salts formed with acids according to the invention include those formed with mineral acids, with strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄) alkyl or aryl sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine.

Especially suitable pharmaceutically acceptable salts of the compounds of the invention are hydrochloric and trifluoroacetic salts.

### Treatment of Cancer

The invention provides a treatment of platinum resistant cancer in a subject or patient, and in particular in the treatment of platinum resistant cancer in a human subject or human patient) using a compound of the invention (for example a compound of formula (I), (Ia), (Ib), (II) or (III)).

Cancer refers to or describes a physiological condition typically characterized by unregulated cell growth. Cancer as described herein includes benign and malignant cancers as well as dormant tumors and micrometastases. It includes tumours of epithelial cells (carcinomas), the blood or bone marrow (leukemias, lymphomas), connective tissues (sarcomas) and cancers in other cells types (for example brain cancers, blastomas).

Examples of cancers include, but are not limited to, ovarian cancer (including epithelial ovarian cancer (for example high-grade serous ovarian cancer) and platinum-resistant ovarian cancer (for example platinum-resistant high-grade serous ovarian cancer)), breast cancer (including platinum-resistant breast cancers), squamous cell cancer, lung cancer (including small-cell lung cancer, non-small cell lung cancer, adenocarcinoma of the lung, squamous carcinoma of the lung and platinum-resistant lung cancers), cancer of the peritoneum, hepatocellular cancer, gastric or stomach cancer (including gastrointestinal cancer), pancreatic cancer (including platinum-resistant pancreatic cancers), glioblastoma, cervical cancer, liver cancer, bladder cancer, hepatoma, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia; acute lymphoblastic leukemia; Hairy cell leukemia; chronic myeloblastic leukemia; and post-transplant lymphoproliferative disorder, as well as abnormal vascular proliferation associated with phakomatoses, edema (such as that associated with brain tumors), and Meigs' syndrome.

The treatments of the present invention are especially useful in the treatment of ovarian cancers including epithelial ovarian cancer (for example high-grade serous ovarian cancer) and platinum-resistant ovarian cancers (for example platinum-resistant high-grade serous ovarian cancer).

The term platinum resistant cancer is defined as cancer that has progressed while receiving chemotherapeutic treatment using a platinum containing drug (i.e. the cancer is platinum refractory), or cancer that has responded to chemotherapeutic treatment using a platinum containing drug, but demonstrates recurrence within a relatively short period of time following the completion of treatment using a platinum containing drug (for example, the cancer has recurred within 18 months, 12 months, 9 months, 6 months, 3 months or 1 month, and in particular within 6 months, of completion of treatment using a platinum containing drug). For example, for ovarian cancer (and in particular for high grade serous ovarian cancer, which is the most common subtype of epithelial ovarian cancer), recurrence within 12 months, and especially within 6 months, of completing initial platinum-based chemotherapy is considered as platinum-resistant cancer according to the present invention. The term platinum resistant cancer also includes cancers in which the tumour contains cells that are resistant to chemotherapeutic treatment using a platinum containing drug. Such cancers have the potential to be platinum refractory (disease progression during therapy) or recur following completing therapy.

The treatments of the present invention are useful in the treatment of platinum resistant cancer in any of the types of cancer described above.

The treatments of the present invention are particularly useful in the treatment of platinum resistant ovarian cancer (for example platinum resistant epithelial ovarian cancer, and in particular platinum-resistant high-grade serous ovarian cancer), testicular cancer, head and neck cancer, lung cancer (for example non-small cell lung cancer), sarcomas, lymphomas, bladder cancer, cervical cancer, breast cancer, mesothelioma and pancreatic cancer, and especially in the treatment of platinum resistant ovarian cancer (for example platinum resistant epithelial ovarian cancer, and in particular platinum-resistant high-grade serous ovarian cancer).

The treatments of the present invention are also particularly useful in the treatment of platinum resistant cancers wherein the majority of the tumour cells are platinum resistant, and cancers wherein the tumours contain both platinum sensitive and platinum resistant cells. The treatments have utility in a patient who is resistant (fully or partially) to treatment using a platinum containing drug.

The treatments of the present invention are also useful in the treatment of cancers that are platinum sensitive, but which are susceptible to recurring in a platinum resistant form, such as ovarian cancer, breast cancer, lung cancer, mesothelioma and pancreatic cancer.

The invention also provides a method for the treatment of a cancer (for example the treatment of a cancer as described above, for example the treatment of a platinum resistant cancer, such as platinum resistant ovarian cancer), comprising administering a compound of the invention (for example a compound of formula (I), (Ia), (Ib), (II) or (III)) to a subject in need thereof. The invention also provides the use of a compound of the invention (for example a compound of formula (I), (Ia), (Ib), (II) or (III)) for the manufacture of a medicament for a treatment described above, for example the treatment of a platinum resistant cancer, such as platinum resistant ovarian cancer

The invention further provides a treatment of cancer in a subject or patient (for example a cancer described above and/or a platinum resistant cancer), and in particular in the treatment of ovarian cancer in a human subject or human patient (for example platinum resistant ovarian cancer), using a compound of the invention (for example a compound of formula (I), (Ia), (Ib), (II) or (III)) in combination with a platinum containing drug (for example cisplatin or carboplatin). Such treatments of the present invention are useful in the treatment of cancers that are platinum sensitive, but which are susceptible to recurring in a platinum resistant form, such as ovarian cancer, breast cancer, lung cancer, mesothelioma and pancreatic cancer.

The invention further provides a method for the treatment of a cancer (for example the treatment of a cancer as described above, such as a cancer that is platinum sensitive but which is at risk of developing platinum resistance), comprising administering a compound of the invention (for example a compound of formula (I), (Ia), (Ib), (II) or (III)) and a platinum containing drug to a subject in need thereof, and a method for the treatment of a cancer (for example the treatment of a cancer as described above), comprising administering a compound of the invention (for example a compound of formula (I), (Ia), (Ib), (II) or (III)) and a platinum containing drug, to a subject in need thereof in a combined treatment.

The invention further provides the use of a compound of the invention (for example a compound of formula (I), (Ia), (Ib), (II) or (III)), in combination with a platinum containing drug, for the manufacture of a medicament for a treatment described above, for example the treatment of a cancer that is platinum sensitive but which is at risk of developing platinum resistance. The invention further provides the use of a compound of the invention (for example a compound of formula (I), (Ia), (Ib), (II) or (III)), in combination with a platinum containing drug, for the manufacture of a medicament for a treatment described above, for example the treatment of a cancer, such as ovarian cancer.

### Definitions

The following definitions apply to the terms as used throughout this specification, unless otherwise limited in specific instances.

As used herein, the term “alkyl” means both straight and branched chain saturated hydrocarbon groups. Examples of alkyl groups include methyl, ethyl, n-propyl, iso-propyl, n-butyl, t-butyl, i-butyl, sec-butyl, pentyl and hexyl groups. Among unbranched alkyl groups, there are preferred methyl, ethyl, n-propyl, n-butyl groups. Among branched alkyl groups, there may be mentioned iso-propyl, t-butyl, i-butyl, 1-ethylpropyl and 1-ethylbutyl groups.

As used herein, the term “cycloalkyl” means a saturated group in a ring system. A cycloalkyl group can be monocyclic or bicyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo [2.2.1]hept-2-yl. Preferably, the cycloalkyl group is monocyclic.

As used herein, the term “halogen” or “halo” means fluorine (fluoro), chlorine (chloro), bromine (bromo) or iodine. Unless otherwise stated, fluorine, chlorine and bromine are particularly preferred.

As used herein, the term “heterocyclyl” means an aromatic or a non-aromatic cyclic group of carbon atoms wherein from one to three of the carbon atoms is/are replaced by one or more heteroatoms independently selected from nitrogen, oxygen or sulfur. A heterocyclyl (or heterocycle) group may, for example, be monocyclic or bicyclic. In a bicyclic heterocyclyl (or heterocycle) group there may be one or more heteroatoms in each ring, or only in one of the rings. A heteroatom may be S, O or N, and is preferably O or N. Heterocyclyl groups containing a suitable nitrogen atom include the corresponding N-oxides.

Examples of monocyclic non-aromatic heterocyclyl include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Examples of bicyclic heterocyclyl groups in which one of the rings is non-aromatic include dihydrobenzofuranyl, indanyl, indolinyl, isoindolinyl, tetrahydroisoquinolinyl, tetrahydroquinolyl and benzoazepanyl.

Examples of monocyclic aromatic heterocyclyl groups include furanyl, thienyl, pyrrolyl, oxazolyl, thiazolyl, imidazolyl, oxadiazolyl, thiadiazolyl, pyridyl, triazolyl, triazinyl, tetrazolyl, pyridazyl, isothiazolyl, isoxazolyl, pyrazinyl, pyrazolyl and pyrimidinyl.

Examples of bicyclic aromatic heterocyclyl groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole. Further examples of bicyclic aromatic heterocyclyl groups include those in which one of the rings is aromatic and the other is non-aromatic, such as dihydrobenzofuranyl, indanyl, indolinyl, isoindolinyl, tetrahydroisoquinolinyl, tetrahydroquinolyl and benzoazepanyl.

The compounds of the invention may contain chiral (asymmetric) centers, or the molecule as a whole may be chiral. For example compounds of formula (I), (Ia), (Ib) or (II) as described above in which Z is CR⁴ have chiral centres at the Z atom. The individual stereoisomers (enantiomers and diastereoisomers) and mixtures of these are within the scope of the present invention.

### Compositions and Doses

The amount of a compound of the invention which is required to achieve a therapeutic effect will vary with the particular compound, the route of administration, the subject under treatment, including the type, species, age, weight, sex, and medical condition of the subject and the renal and hepatic function of the subject, and the particular disorder or disease being treated, as well as its severity. An ordinarily skilled physician can readily determine and prescribe the effective amount of the drug required to prevent, counter or arrest the progress of the condition for which the compound of the invention is administered, for example for the treatment of cancer.

A medicament typically contains from about 0.01 mg to about 500 mg of the active ingredient (i.e. a compound of the invention), for example from about 0.1 mg to about 100 mg of active ingredient, or 1 mg to about 100 mg of active ingredient.

Oral dosages of a compound of the present invention, when used for the indicated diseases, will range between about 0.001 mg per kg of body weight per day (mg/kg/day) to about 10 mg/kg/day of a compound of the invention, for example 0.01 to 5 mg/kg/day, 0.1 to 5 mg/kg/day or 0.5 to 3.0 mg/kg/day for adult humans. An oral daily dosage therefore ranges from 0.05 mg to about 700 mg of a compound of the invention, for example 0.5 mg to 350 mg, 5 mg to 350 mg, or 25 to 250 mg, for example 0.05, 0.1, 0.5, 1, 5, 10, 15, 20, 25, 50, 75, 100, 125, 150, 175, 200, 225 or 250 mg, for adult humans.

A compound of the invention may be administered in a single daily dose, or the total daily dosage may be administered in divided doses of two, three or four times daily. For oral administration, the compositions are preferably provided in the form of tablets or other forms of presentation provided in discrete units containing from about 0.01 mg to about 500 mg of the compound of the invention, for example from about 0.1 mg to about 100 mg, from about 1 mg to about 100 mg of active ingredient, for example 0.1, 0.5, 1.0, 2.5, 5.0, 10.0, 15.0, 25.0, 50.0, 100, or 500 milligrams of the active ingredient.

Intravenously, the most preferred doses will range from about 0.001 to about 10 mg/kg/minute during a constant rate infusion, for example 0.01 to 5 mg/kg/day, 0.1 to 5 mg/kg/day or 0.5 to 3.0 mg/kg/day during a constant rate infusion.

While it is possible for the active ingredient to be administered alone, it is preferable for it to be present in a pharmaceutical formulation or composition. Accordingly, the invention provides a pharmaceutical formulation or composition comprising a compound according to the invention, and a pharmaceutically acceptable diluent, excipient or carrier (collectively referred to herein as “carrier” materials). Pharmaceutical compositions of the invention may take the form of a pharmaceutical formulation as described below.

Pharmaceutical formulations of a compound of the invention include those suitable for oral, parenteral (including subcutaneous, intradermal, intramuscular, intravenous [bolus or infusion], intraperitoneal and intraarticular), inhalation (including fine particle dusts or mists which may be generated by means of various types of metered dose pressurized aerosols), nebulizers or insufflators, rectal and topical (including dermal, buccal, sublingual, and intraocular) administration.

The formulations of a compound of the invention may conveniently be presented in unit dosage form and may be prepared by any of the methods well known in the art of pharmacy.

Formulations of a compound of the invention suitable for oral administration may be presented as discrete units such as capsules, cachets, pills or tablets each containing a predetermined amount of the active ingredient; as a powder or granules; as a solution or a suspension in an aqueous liquid or a non-aqueous liquid, for example as elixirs, tinctures, suspensions or syrups; or as an oil-in-water liquid emulsion or a water-in-oil liquid emulsion. The active ingredient may also be presented as a bolus, electuary or paste.

Formulations for parenteral administration, in particular for intravenous and intraperitoneal administration, include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations of a compound of the invention are those containing an effective dose, as hereinbefore recited, or an appropriate fraction thereof, of the compound of the invention.

It should be understood that in addition to the ingredients particularly mentioned above, the formulations for use in the invention may include other agents conventional in the art having regard to the type of formulation in question, for example those suitable for oral administration may include flavouring agents.

Whilst a compound of the invention may be used as the sole active ingredient in a medicament, it is also possible for the compound to be used in combination with one or more further therapeutic agents. Thus, the invention also provides a compound according to the invention together with a further therapeutic agent, for simultaneous, sequential or separate administration.

### Platinum Containing Drugs

The compounds and the treatments of the present invention may be used in combination with a platinum containing drug.

The invention further provides a composition (for example a pharmaceutical composition) comprising a compound of the invention (for example, a compound of formula (I), (Ia), (Ib), (II) or (III)) and a platinum containing drug, and optionally a pharmaceutically suitable carrier.

The invention further provides a kit comprising a compound of the invention (for example, a compound of formula (I), (Ia), (Ib), (II) or (III)) and a platinum containing drug.

Various platinum containing compounds are known which may be used as anti-cancer drugs, in particular for the treatment of ovarian cancer, testicular cancer, head and neck cancer, lung cancer, sarcomas, lymphomas, bladder cancer, cervical cancer, breast cancer, mesothelioma and pancreatic cancer.

A platinum containing drug (also known as a platinum containing drug anti-cancer drug or a platinum-based antineoplastic drug) comprises an organic compound which contains platinum as an integral part of the molecule. Platinum containing drugs include cisplatin, carboplatin, oxaliplatin, satraplatin, picoplatin, nedaplatin, triplatin, tetraplatin, ormaplatin, phosphaplatin and lipoplatin (a liposomal version of cisplatin). Preferably, the platinum containing drug is carboplatin or cisplatin.

In embodiments in which the treatment comprises administration of a compound of the invention and platinum containing drug, the platinum containing drug may be selected from the group consisting of cisplatin, carboplatin, oxaliplatin, satraplatin, picoplatin, nedaplatin, triplatin, tetraplatin, ormaplatin, phosphaplatin and lipoplatin. Preferably, the platinum containing drug is cisplatin or carboplatin. The selection of the platinum containing drug may be dependent on factors including the type of cancer and the health of the patient. For example, for the treatment of ovarian cancer, the treatment of the present invention may comprise a compound of the invention and platinum containing drug, wherein preferably the platinum containing drug is carboplatin or cisplatin.

The pharmaceutical formulation of the platinum containing drug for use in the invention is preferably a formulation for intravenous administration (for example in the case of cisplatin, carboplatin, and oxaliplatin) or oral administration (for example in the case of satraplatin).

The optimal dose of the platinum containing drug depends on the dosing schedule, the potency of the particular drug chosen, the age, size, sex and condition of the patient, the nature and severity of the disease, and other relevant medical and physical factors. Thus, the pharmaceutically effective amount can be readily determined by the caregiver or clinician. The platinum containing drug may be used, for example, in the amounts indicated in the Physicians' Desk Reference (PDR) or as otherwise determined by one of ordinary skill in the art.

Generally, an appropriate amount of platinum containing drug is chosen to obtain a chemotherapeutic effect. For an intravenous dose of a platinum containing drug the typical infusion rate is in the range from about 0.005 to about 0.05 mg/kg/minute. An effective intravenous dose of platinum containing drug is typically from about 0.1 to about 50 mg/kg of body weight and preferably about 1 to about 5 mg/kg of body weight. For example, in embodiments where the platinum containing drug is cisplatin, the intravenous dose of the of cisplatin will typically contain from about 10 mg/m² to about 400 mg/m² of the active ingredient, preferably from about 50 mg/m² to about 200 mg/m² of active ingredient, for example 75 mg/m² to 100 mg/m² (corresponding to a dosage of about 125-185 mg a day). For example, in embodiments where the platinum containing drug is carboplatin, the intravenous dose of the of carboplatin will typically contain from about 100 mg/m² to about 600 mg/m² of the active ingredient, preferably from about 200 mg/m² to about 400 mg/m² of active ingredient, for example 300 mg/m² to 360 mg/m² (corresponding to a dosage of about 500-600 mg a day).

A compound of the invention and platinum containing drug for use in the present invention may be administered simultaneously, sequentially or separately. The platinum containing drug is typically present in a therapeutically effective amount.

A platinum containing drug is typically administered on day one of a cycle, preferably a cycle lasting 20 to 30 days, for example a 21 day or 28 day cycle. On the days of the cycle after day one, no platinum containing drug is typically administered. A compound of the invention may be administered simultaneously, sequentially or separately to the platinum containing drug on day one of a cycle. A compound of the invention may be administered on one or more further days of the cycle, for example every seventh day of the cycle, every second day of the cycle or every day of the cycle. The cycle may be repeated two or more times, for example 4 times, 6 times or 8 times.

The active agents (for example the compound of the invention and/or platinum containing drug and/or any further therapeutic agent) in the present invention may be administered by the same or different routes of administration.

### Further Therapeutic Agents

In addition to, or instead of, a platinum-containing drug, the treatments of the present invention may comprise the administration of one or more further therapeutic agent. A therapeutic agent is a chemical compound useful in the treatment of a disease. The further therapeutic agent may be, for example, a chemotherapeutic agent, a radiotherapeutic agent, another compound of the invention, an AKT inhibitor, or a dual AKT/DNA-PK inhibitor. The treatments of the present invention may be used in combination with surgery for treatment of the cancer.

A compound of the invention and further therapeutic agent may be administered simultaneously sequentially or separately. The further therapeutic agent is typically present in a therapeutically effective amount.

The invention further provides a composition (for example a pharmaceutical composition) comprising a compound of the invention (for example, a compound of formula (I), (Ia), (Ib), (II) or (III)) and a further therapeutic agent, and optionally a pharmaceutically suitable carrier.

The invention further provides a kit comprising a compound of the invention (for example, a compound of formula (I), (Ia), (Ib), (II) or (III)) and a further therapeutic agent.

A chemotherapeutic agent is a chemical compound useful in the treatment of cancer. A chemotherapeutic agent according to the present invention may be an alkylating agent, anthracycline, cytoskeletal disruptor (Taxane), epothilone, histone deacetylase inhibitor, kinase inhibitor, poly ADP ribose polymerase (PARP) inhibitor, mitotic inhibitor, monoclonal antibody, nucleotide analogue or precursor, peptide antibiotic, retinoid, vinca alkaloid or derivative, or immuno-oncology agents.

Examples of chemotherapeutic agents include: Abitrexate (Methotrexate Injection), Osteosarcoma, Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMETREXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), DexpakTaperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab Injection), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Imbruvica (Ibrutinib), Imfinzi (Durvalumab), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Keytruda (Pembrolizumab), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lynparza (Olaparib), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Opdivo (Nivolumab), Panretin (Alitretinoin), Perjeta (Pertuzumab Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Rubraca (Rucaparib), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zejula (Niraparib), Zelboraf (Vemurafenib), Zevalin (lbritumomab Tiuxetan), Zoladex (Goserelin), Zolinza (Vorinostat), Zometa (Zoledronic acid), Zortress (Everolimus), Zytiga (Abiraterone); and pharmaceutically acceptable salts, acids or derivatives of any of the above.

Examples of immuno-oncology agents include checkpoint inhibitors, for example an agent or antibody that inhibits one or more of CTLA4, PD-1, PD-L1, LAG-3, B7-H3, B7-H4, TIM3, VISTA and KIR. In particular, the checkpoint inhibitor may be an agent or antibody that inhibits PD-1 or PD-L1, for example PD-1 inhibitors (including anti-PD antibodies) and PD-L1 inhibitors (including anti-PD-L1 antibodies). Examples of PD-1 inhibitors include cemiplimab, nivolumab and pembrolizumab. Examples of PD-L1 inhibitors include atezolizumab, avelumab and durvalumab.

The treatment of the present invention may comprise a compound of the invention and one further therapeutic agent. The treatment of the present invention may comprise a compound of the invention and two further therapeutic agents, for example a compound of the invention and two further therapeutic agents wherein one further therapeutic agent is a platinum containing drug and the other further therapeutic agent is a second chemotherapeutic drug (for example a drug selected from the list in the previous paragraph), a second compound of the invention, a AKT inhibitor, or a dual AKT/DNA-PK inhibitor. For the treatment of ovarian cancer, the treatment of the present invention may comprise a compound of the invention and one or more further therapeutic agent, for example a platinum containing drug, wherein preferably the platinum containing drug is carboplatin or cisplatin, and/or a mitotic inhibitor, wherein preferably the mitotic inhibitor is paclitaxel.

The above other therapeutic agents, may be used, for example, in those amounts indicated in the Physicians' Desk Reference (PDR) or as otherwise determined by one of ordinary skill in the art.

### AKT and DNA-PK Inhibitors

The compounds of the invention (for example, a compound of formula (I), (Ia), (Ib), (II) or (III)) have activity as inhibitors of the enzyme AKT. An AKT inhibitor is a compound capable of binding to an AKT enzyme site and blocking the actions of an AKT enzyme. The compounds of invention (for example, a compound of formula (I), (Ia), (Ib), (II) or (III)) also have activity as inhibitors of the enzyme DNA-PK. A DNA-PK inhibitor is a compound capable of binding to the DNA-PK enzyme site and blocking the actions of DNA-PK.

A compound is considered to be a dual AKT/DNA-PK inhibitor if it inhibits an AKT enzyme with a pIC₅₀ of 6.0 or greater and inhibits a DNA-PK enzyme with a pIC₅₀ of 6.0 or greater. As the compound of the invention have activity as inhibitors of the enzyme AKT and are capable of binding to a DNA-dependent protein kinase (DNA-PK) enzyme site and blocking the actions of DNA-PK enzyme, the compounds of the invention are dual AKT/DNA-PK inhibitors.

A compound is considered to be an inhibitor of AKT if it inhibits an AKT enzyme with a pIC₅₀ of 6.0 or greater. In certain embodiments, preferred compounds of the invention have a pIC₅₀ of 7.0 or greater, 7.5 or greater, 8.0 or greater, 8.5 or greater, or 9.0 or greater for an AKT enzyme. In one preferred embodiment, preferred compounds of the invention have a pIC₅₀ of 8.0 or greater, 8.5 or greater, or 9.0 or greater for an AKT enzyme. At the time of writing, three members of the AKT family have been discovered: AKT1, AKT2 and AKT3. A compound is considered to be an inhibitor of AKT if it inhibits at least one AKT enzyme with the stated pIC₅₀. Accordingly, compounds of the invention have a pIC₅₀ of 6.0 or greater for AKT1 and/or AKT2 and/or AKT3. In certain preferred embodiments, compounds of the invention have a pIC₅₀ of 7.0 or greater for AKT1 and/or AKT2 and/or AKT3. In certain preferred embodiments, compounds of the invention have a pIC₅₀ of 8.0 or greater for AKT1 and/or AKT2 and/or AKT3. In certain preferred embodiments, compounds of the invention have a pIC₅₀ of 8.5 or greater for AKT1 and/or AKT2 and/or AKT3. In certain preferred embodiments, compounds of the invention have a pIC₅₀ of 9.0 or greater for AKT1 and/or AKT2 and/or AKT3.

Certain preferred compounds of the invention inhibit at least two AKT enzymes with the stated pIC₅₀. Accordingly, preferred compounds of the invention have a pIC₅₀ of 6.0 or greater for at least two of AKT1, AKT2 and AKT3. As such, in certain preferred embodiments, compounds of the invention have a pIC₅₀ of 7.0 or greater for at least two of AKT1, AKT2 and AKT3. In certain preferred embodiments, compounds of the invention have a pIC₅₀ of 8.0 or greater for at least two of AKT1, AKT2 and AKT3. In certain preferred embodiments, compounds of the invention have a pIC₅₀ of 8.5 or greater for at least two of AKT1, AKT2 and AKT3. In certain preferred embodiments, compounds of the invention have a pIC₅₀ of 9.0 or greater for at least two of AKT1, AKT2 and AKT3.

Certain preferred compounds inhibit at least three AKT enzymes with the stated pIC₅₀. Accordingly preferred compounds of the invention have a pIC₅₀ of 6.0 or greater for AKT1 and pIC₅₀ of 6.0 or greater for AKT2 and pIC₅₀ of 6.0 or greater for AKT3. As such, in certain preferred embodiments, compounds of the invention have a pIC₅₀ of 7.0 or greater for AKT1 and AKT2 and AKT3. In certain preferred embodiments, compounds of the invention have a pIC₅₀ of 8.0 or greater for AKT1 and AKT2 and AKT3. In certain preferred embodiments, compounds of the invention have a pIC₅₀ of 8.5 or greater for AKT1 and AKT2 and AKT3. In certain preferred embodiments, compounds of the invention have a pIC₅₀ of 9.0 or greater for AKT1 and AKT2 and AKT3.

Compounds of the present invention are also capable of binding to a DNA-dependent protein kinase (DNA-PK) enzyme site and blocking the actions of a DNA-PK enzyme. A compound is considered to be an inhibitor of DNA-PK if it inhibits a DNA-PK enzyme with a pIC₅₀ of 6.0 or greater. Accordingly, compounds of the invention have a pIC₅₀ of 6.0 or greater for DNA-PK. In certain preferred embodiments, compounds of the invention have a pIC₅₀ of 7.0 or greater for DNA-PK. In certain preferred embodiments, compounds of the invention have a pIC₅₀ of 8.0 or greater DNA-PK. In certain preferred embodiments, compounds of the invention have a pIC₅₀ of 8.5 or greater for DNA-PK. In certain preferred embodiments, compounds of the invention have a pIC₅₀ of 9.0 or greater for DNA-PK.

In certain preferred embodiments, compounds of the invention are dual AKT/DNA-PK inhibitor that inhibit an AKT enzyme with a pIC₅₀ of 7.5 or greater and inhibits a DNA-PK enzyme with a pIC₅₀ of 7.5 or greater. In certain preferred embodiments, compounds of the invention are dual AKT/DNA-PK inhibitor that inhibit an AKT enzyme with a pIC₅₀ of 8.0 or greater and inhibits a DNA-PK enzyme with a pIC₅₀ of 7.5 or greater. In certain preferred embodiments, compounds of the invention are dual AKT/DNA-PK inhibitor that inhibit an AKT enzyme with a pIC₅₀ of 8.0 or greater and inhibits a DNA-PK enzyme with a pIC₅₀ of 8.0 or greater. In certain preferred embodiments, compounds of the invention are dual AKT/DNA-PK inhibitor that inhibit an AKT enzyme with a pIC₅₀ of 8.5 or greater and inhibits a DNA-PK enzyme with a pIC₅₀ of 8.5 or greater. In certain preferred embodiments, compounds of the invention are dual AKT/DNA-PK inhibitor that inhibit an AKT enzyme with a pIC₅₀ of 9.0 or greater and inhibits a DNA-PK enzyme with a pIC₅₀ of 9.0 or greater.

As mentioned above, the compounds of the invention have activity as inhibitors of both AKT and DNA-PK. The compounds of the invention may be competitive inhibitors or partial competitive inhibitors of one or more AKT enzyme. The compounds of the invention may also be competitive inhibitors or partial competitive inhibitors of the DNA-PK enzyme.

### EXAMPLES

### 1. Synthesis of Example Compounds

### Chemistry Materials and Methods

Numerous synthetic routes to the compounds of the present invention can be devised by a person skilled in the art and the exemplified synthetic routes described below do not limit the invention. A number of possible synthetic routes are exemplified below. Where appropriate, any initially produced compound according to the invention can be converted into another compound according to the invention by known methods.

Unless stated otherwise, commercially available compounds were purchased from Sigma Aldrich. Commercially available compounds were used without further purification.

### LC-MS

Compounds were purified and analysed on an LC-MS system (Agilent 6310 Ion Trap LC/MS or Shimadzu LCMS-2010 EV (single quad)) as detailed in Table 1. Compounds were separated over a gradient of methanol in water (5-98% over 12 minutes then 98% methanol for 3 minutes), both containing 0.1% formic acid, or starting from 20 or 50% methanol for the same time periods; or as indicated in the experimental section below.

### NMR

¹H NMR spectra were recorded at room temperature on either a Varian Unity Inova 400 (400 MHz) or a Varian Unity Inova 500 (500 MHz) machine as detailed in the experimental procedures below and Table 1 below. Data are presented as follows: chemical shift in ppm, integration, multiplicity (br=broad, app=apparent, s=singlet, d=doublet, t=triplet, q=quartet, p=pentet, m=multiplet) and coupling constants in Hz.

### HPLC

Where indicated, compounds were separated by high performance liquid chromatography using a Shimadzu HPLC model LC20AD/SPD-M20A (Softa 400 ELSD).

### Optical Rotation

Where indicated, optical rotation of compounds was measured using a Jasco P-2000 polarimeter (Serial number: A087561232, faraday cell: flint glass).

### General Method I: Formation of 2-[4-(3-bromo-1H-pyrazolo[3,4-d]pyrimidin-4-yl)piperazin-1-yl]-2-(4-chlorophenyl)-N,N-dimethyl-ethanamine (Example 1)

### Intermediate 1: methyl 2-bromo-2-(4-chlorophenyl)acetate

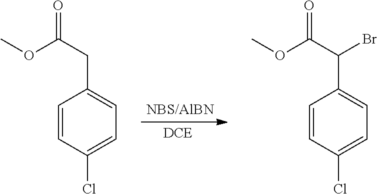

To a stirred solution of methyl 2-(4-chlorophenyl) acetate (5 g, 27.08 mmol) in EDC (50 mL) under N₂ atmosphere was added NBS (5.01 g, 28.17 mmol) followed by AIBN (0.44 g, 2.70 mmol) at room temperature (RT). The resulting reaction mixture was stirred for 3 h at reflux temperature; progress of the reaction was monitored by TLC. The reaction mixture was cooled to RT; diluted with DCM and washed with saturated NaHCO₃ solution. The combined organic extracts were dried over anhydrous Na₂SO₄ and concentrated under reduced pressure to afford Intermediate 1 (5 g, crude) as orange thick syrup. The crude Intermediate 1 was used in the preparation of Intermediate 2 without further purification.

¹H NMR (500 MHz, CDCl₃): δ 7.48 (d, 2H), 7.32 (d, 2H), 5.35 (s, 1H), 3.79 (s, 3H).

### Intermediate 2: tert-butyl 4-[1-(4-chlorophenyl)-2-methoxy-2-oxo-ethyl]piperazine-1-carboxylate

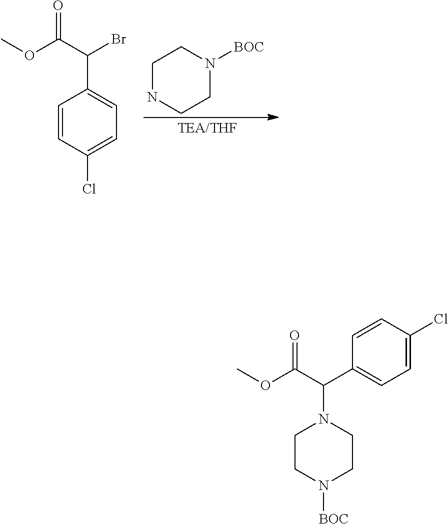

To a stirred solution of Intermediate 1 (0.5 g, 1.89 mmol; crude) in dry THF (5 mL) under N₂ atmosphere were added tert-butyl piperazine-1-carboxylate (0.35 g, 1.89 mmol) and Et₃N (0.818 mL, 5.88 mmol) at RT and the reaction mixture was stirred for 2 h. After consumption of the starting material (monitored by TLC), the reaction mixture was diluted with EtOAc and washed with water. The organic layer was dried over anhydrous Na₂SO₄, filtered and concentrated under reduced pressure to get crude material. The crude material was purified by silica gel column chromatography eluting with 6% MeOH/CH₂Cl₂ and then re-purified by silica gel column chromatography eluting with 40% EtOAc/Hexane to afford Intermediate 2 (0.8 g, 85%) as pale-yellow thick syrup.

TLC: 10% EtOAc/Hexane (R_{f}: 0.45)

¹HNMR (400 MHz, CDCl₃): δ 7.38-7.32 (m, 4H), 4.02 (s, 1H), 3.69 (s, 3H), 3.46-3.39 (m, 4H), 2.43-2.38 (m, 4H), 1.49 (s, 9H).

LC-MS: m/z 269.2 [M-Boc] at 4.09 RT (98.18% purity) (Column: X-select C-18 (50×3.0 mm x 3.5p); 5 mM NH₄OAc: ACN; 0.8 ml/min)

### Intermediate 3: 2-(4-tert-butoxycarbonylpiperazin-1-yl)-2-(4-chlorophenyl)acetic acid

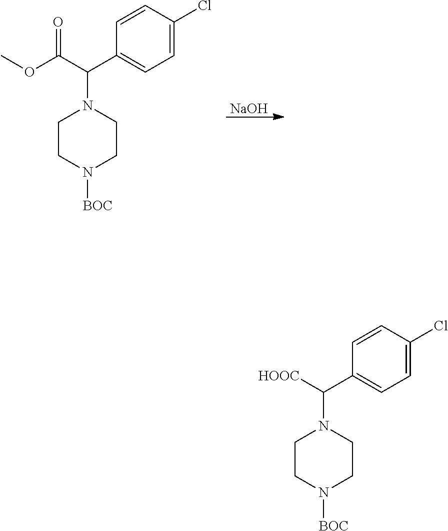 [SMILES: COC(=O)C(c1ccc(Cl)cc1)N1CCN(C(=O)OC(C)(C)C)CC1 ; "NaOH" ; CC(C)(C)OC(=O)N1CCN(C(C(=O)O)c2ccc(Cl)cc2)CC1]

To a stirred solution of Intermediate 2 (0.8 g, 2.18 mmol) in MeOH:H₂O (8 mL, 1:1) was added NaOH (0.168 g, 4.20 mmol) at 0° C. under inert atmosphere. The resulting reaction mixture was allowed to warm to RT and stirred for 12 h; progress of the reaction was monitored by TLC. The reaction mixture was then neutralized to pH^{˜}7 using acetic acid and extracted with EtOAc. The combined organic extracts were dried over anhydrous Na₂SO₄ and concentrated under reduced pressure to afford Intermediate 3 (0.6 g, crude) as white solid. Intermediate 3 was used directly for the synthesis of Intermediate 4.

TLC: 40% EtOAc/Hexane (R_{f}: 0.4)

¹HNMR (400 MHz, DMSO-ds): δ 12.47 (bs, 1H), 7.42-7.39 (m, 4H), 4.02 (s, 1H), 3.36-3.32 (m, 4H), 2.39-2.35 (m, 4H), 1.34 (s, 9H).

LC-MS: m/z 355.3 [M+2]⁺ at 2.56 RT (97.64% purity) (Column: X-select C-18 (50×3.0 mm x 3.5μ); 5 mM NH₄OAc: ACN; 0.8 ml/min)

### Intermediate 4: tert-butyl 4-(1-(4-chlorophenyl)-2-(dimethylamino)-2-oxoethyl)piperazine-1-carboxylate

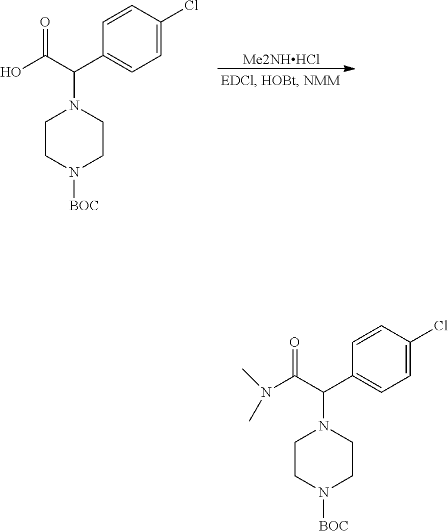 [SMILES: "Me2NH·HCl" ; CC(C)(C)OC(=O)N1CCN(C(C(=O)O)c2ccc(Cl)cc2)CC1 ; CN(C)C(=O)C(c1ccc(Cl)cc1)N1CCN(C(=O)OC(C)(C)C)CC1]

A stirred solution of Intermediate 3 (0.7 g, 1.98 mmol) in dry CH₂Cl₂ (35 mL) under N₂ atmosphere was cooled to 0° C., and EDCl.HCl (1.13 g, 5.95 mmol), HOBt.H₂O (805 mg, 5.95 mmol) were added, followed by NMM (1.3 mL, 11.91 mmol) and N,N-dimethylamine hydrochloride (0.64 mL, 7.94 mmol). The reaction mixture was stirred at RT for 16 h. After consumption of the starting material (monitored by TLC), the reaction was diluted with CH₂Cl₂ and washed with water. The organic layer was dried over anhydrous Na₂SO₄, filtered and concentrated under reduced pressure to give the crude material. The crude material was purified by silica gel column chromatography eluting with 4% MeOH/CH₂Cl₂ to afford Intermediate 4 (0.45 g, 60%) as pale-yellow syrup.

TLC: 10% MeOH/CH₂Cl₂ (R_{f}: 0.7)

¹HNMR (500 MHz, CDCl₃): δ 7.39-7.29 (m, 4H), 4.28 (s, 1H), 3.43-3.41 (m, 4H), 2.98 (s, 3H), 2.96 (s, 3H), 2.51-2.42 (m, 4H), 1.42 (s, 9H)

LC-MS: m/z 382 [M+2]⁺ at 4.10 RT (94.65% purity) (Column: X-bridge C-18 (50×3.0 mm×3.5μ); 5 mM NH₄OAc: ACN; 0.8 ml/min)

### Intermediate 5: tert-butyl 4-(1-(4-chlorophenyl)-2-(dimethylamino) ethyl) piperazine-1-carboxylate

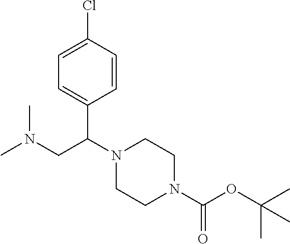 [SMILES: CN(C)CC(c1ccc(Cl)cc1)N1CCN(C(=O)OC(C)(C)C)CC1]

To a stirred solution of Intermediate 4 (0.85 g, 2.64 mmol) in dry THF (8.5 mL) was added BH₃.DMS (6.37 mL, 5M in Et₂O) at 0° C. under inert atmosphere. The resulting reaction mixture was allowed to warm to RT and stirred for 3 h. The mixture was then cooled to 0° C., and H₂O:THF (8.5 mL 1:1), 2N NaOH (8.5 mL) and 30% H₂O₂ (8.5 mL) were added. Stirring was continued for another 15 min at RT. Progress of the reaction was monitored by TLC and once the reaction was complete the reaction mixture was diluted with water and extracted with EtOAc. The organic layer was dried over anhydrous Na₂SO₄, filtered and concentrated under reduced pressure. The obtained crude material was purified by silica gel column chromatography eluting with 40% EtOAc/Hexane to afford Intermediate 5 (0.5 g, 61%) as off white solid.

TLC: 10% MeOH/DCM (R_{f}: 0.25)

### Intermediate 6: 2-(4-chlorophenyl)-N,N-dimethyl-2-(piperazin-1-yl)ethanamine hydrochloride

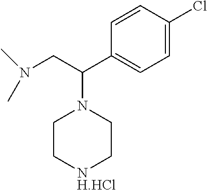

To a stirred solution of Intermediate 5 (400 mg, 1.05 mmol) in MeOH (3.2 mL) was added 4N HCl in 1,4-Dioxane (3.2 mL) at 0° C. under inert atmosphere. The resulting reaction mixture was allowed to warm to RT and stirred for 2 h; progress of the reaction was monitored by TLC. The reaction mixture was then concentrated under reduced pressure to afford Intermediate 6 (335 mg, crude) as an off-white solid. Intermediate 6 was used directly for synthesis of Example 1 without further purification.

### Intermediate 7: 1-(tert-butyl)-4-chloro-3-(trifluoromethyl)-1H-pyrazolo[3,4-d]pyrimidine

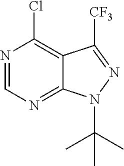 [SMILES: CC(C)(C)n1nc(C(F)(F)F)c2c(Cl)ncnc21]

Step 1: A stirred solution of ethyl 2-cyanoacetate (10 g, 0.088 mol) in dry DCM (100 mL) was cooled to 0° C. under N₂ atmosphere; to this trifluoroacetic anhydride (14.86 mL, 0.106 mol) followed by Et₃N (36.86 mL, 0.265 mol) was added and stirred at RT for 1 h. After consumption of the starting material (monitored by TLC), the reaction was diluted with CH₂Cl₂ and washed with saturated NaHCO₃. The organic layer was dried over anhydrous Na₂SO₄, filtered and concentrated under vacuum. Obtained crude material was purified by silica gel column chromatography eluting with 6% MeOH/CH₂Cl₂ to afford ethyl 2-cyano-4, 4, 4-trifluoro-3-oxobutanoate (18 g, 54%) as an orange thick syrup.

TLC: 10% MeOH/CH₂Cl₂ (R_{f}: 0.25)

¹HNMR (400 MHz, CDCl₃): δ 4.18-4.07 (m, 1H), 3.18-3.12 (m, 2H), 1.28 (t, 3H)

LC-MS: m/z 208.1 [M−1] at 1.63 RT (95.70% purity)

Step 2: To a stirred solution of ethyl 2-cyano-4, 4, 4-trifluoro-3-oxobutanoate (1.8 g, 8.6 mmol) in dimethylcarbonate (16.3 mL) were added tert-butylhydrazine hydrochloride (Ia) (1.93 g, 15.5 mmol) followed by trifluoroacetic acid (1.34 mL, 17.6 mmol) and dried molecular sieves (2.7 g). The resulting reaction mixture was heated to 80° C. and stirred for 18 h. After consumption of the starting material (monitored by TLC), the reaction mixture was filtered through a pad of celite and the bed was washed with EtOAc. Obtained filtrate was washed with 5% NaHCO₃, 5% NaOH, brine and water. The organic layer was dried over anhydrous Na₂SO₄, filtered, concentrated under reduced pressure; which was purified by silica gel column chromatography eluting with 15% EtOAc/Hexane to afford ethyl 5-amino-1-(tert-butyl)-3-(trifluoromethyl)-1H-pyrazole-4-carboxylate (0.25 g, 10.4%) as pale yellow syrup.

TLC: 5% MeOH/CH₂Cl₂ (R_{f}: 0.5)

¹HNMR (400 MHz, CDCl₃): δ 5.47 (bs, 2H), 4.28 (q, 2H), 1.64 (s, 9H), 1.38 (t, 3H)

Step 3: To a stirred solution of ethyl 5-amino-1-(tert-butyl)-3-(trifluoromethyl)-1H-pyrazole-4-carboxylate (0.25 g, 0.89 mmol) in formamide (0.75 mL) was added ammonium carbonate (0.104 g, 10.8 mmol) under N₂ atmosphere. The reaction mixture was heated to 170° C. and stirred for 52 h. After consumption of the starting material (monitored by TLC), the reaction mixture was diluted with EtOAc and washed with water. The organic layer was dried over anhydrous Na₂SO₄, filtered and concentrated under reduced pressure. Obtained crude material was purified by silica gel column chromatography eluting with 30% EtOAc/Hexane to afford 1-(tert-butyl)-3-(trifluoromethyl)-1H-pyrazolo[3,4-d]pyrimidin-4-ol (0.1 g, 43%) as an off-white solid.

TLC: 20% EtOAc/Hexane (R_{f}: 0.3)

¹HNMR (500 MHz, CDCl₃): δ 11.81 (bs, 1H), 8.02 (s, 1H), 1.84 (s, 9H)

LC-MS: m/z 258.6 [M−1] at 3.83 RT (84.09% purity)

Step 4: to a stirred solution of 1-(tert-butyl)-3-(trifluoromethyl)-1H-pyrazolo[3,4-d]pyrimidin-4-ol (0.1 g, 0.35 mmol) in SOCl₂ (1 mL) was added DMF (0.05 mL) at RT under N₂ atmosphere. The resulting reaction mixture was heated to 75° C. and stirred for 2 h. After consumption of the starting material (by TLC), the volatiles were removed under reduced pressure. Obtained residue was diluted with EtOAc and washed with saturated NaHCO₃ solution. The organic layer was dried over anhydrous Na₂SO₄, filtered and concentrated under reduced pressure to get crude; which was purified by silica gel column chromatography eluting with 6% EtOAc/Hexane to afford Intermediate 7 (0.05 g, 46%) as an off-white solid.

TLC: 40% EtOAc/Hexane (R_{f}: 0.8)

¹HNMR (400 MHz, CDCl₃): δ 8.81 (s, 1H), 1.89 (s, 9H)

LC-MS: m/z 278.9 [M+1]⁺ at 4.33 RT (86.48% purity)

### Intermediate 8: 4-Chloro-3-methyl-1H-pyrazolo [3, 4-d] pyrimidine

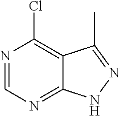

Step 1: To a stirred solution of POCl₃ (80 mL, 0.428 mol) at 0° C. under N₂ atmosphere was added DMF slowly and stirred for 10 min at RT. To this pyrimidine-4,6-diol (20 g, 0.089 mol) was added portion wise at 0° C. and stirred for 3 h at reflux temperature. The resulting reaction mixture was cooled to RT and stirring was continued for another 16 h at RT. After consumption of starting material (by TLC), to the reaction crushed ice was added and extracted with EtOAc. The organic layer was dried over anhydrous Na₂SO₄, filtered and concentrated under reduced pressure to afford 4, 6-dichloropyrimidine-5-carbaldehyde (20 g, crude) as yellow solid. This material was directly used for the next step without further purification.

TLC: 40% EtOAc/Hexane (R_{f}: 0.75)

¹H NMR (500 MHz, CD₃OD): δ 8.22 (s, 2H), 7.2-7.4 (m, 5H), 5.99 (d, 1H), 4.8 (m, 1H), 4.6 (s, 2H), 4.35 (s, 1H), 4.2 (s, 1H), 4.0 (m, 1H), 3.95 (d, 1H), 3.7 (d, 1H), 2.25 (m, 1H), 2.0 (m, 1H), 1.9-1.6 (m, 4H).

Mass (ESI): 442.6 (M⁺+1).

Step 2: To a stirred solution of 4, 6-dichloropyrimidine-5-carbaldehyde (27 g, 0.015 mol) in dry ether (270 mL) at 0° C. under N₂ atmosphere was added methyl magnesium bromide (1M, 166.7 mL, 0.016 mol) slowly. The reaction mixture was stirred for 3 h at RT. After consumption of starting material (by TLC), the reaction was quenched with saturated NH₄Cl solution and aqueous layer was extracted with Ether. The organic layer was dried over anhydrous Na₂SO₄, filtered and concentrated under reduced pressure. Obtained crude material was purified by silica gel column chromatography eluting with 15% EtOAc/Hexane to afford 1-(4, 6-dichloropyrimidin-5-yl) ethanol (20 g, 68%) as yellow solid.

TLC: 30% EtOAc/Hexane (R_{f}: 0.2)

¹H NMR (500 MHz, CDCl₃): δ 8.4 (s, 1H), 7.8 (s, 1H), 7.4-7.2 (m, 5H), 6.8 (d, 1H), 5.85 (m, 1H), 5.75 (br s, 1H), 5.2 (m, 1H), 5.15 (m, 1H), 4.8-4.6 (m, 3H), 4.55 (s, 1H), 4.0-3.9 (m, 2H), 3.8 (t, 1H), 2.35 (m, 1H), 2.0-1.8 (m, 2H), 1.79 (m, 2H), 1.65 (s, 3H), 1.6 (m, 1H), 1.4 (s, 3H).

HPLC(Purity): 99.54%

Step 3: To a stirred solution of 1-(4, 6-dichloropyrimidin-5-yl)ethanol (20 g, 0.1 mol) in acetone (320 mL) at 0° C. under N₂ atmosphere was added CrO₃ (20.6 g, 0.2 mol) portion wise at 0° C. The reaction mixture was stirred at RT for 3 h. After consumption of the starting material (by TLC), IPA (34 mL) was slowly added to reaction and further stirred for 15 min. The reaction was diluted with CH₂Cl₂ and quenched with saturated NaHCO₃ solution. The resulting mass was filtered through a pad of celite; obtained filtrate was dried over anhydrous Na₂SO₄, filtered and concentrated under vacuum. The crude material was purified by silica gel column chromatography eluting with 15% EtOAc/Hexane to afford 1-(4, 6-dichloropyrimidin-5-yl) ethanone (15.1 g, 76.3%) as yellow solid.

TLC: 30% EtOAc/Hexane (R_{f}: 0.7)

Step 4: To a stirred solution of 1-(4, 6-dichloropyrimidin-5-yl)ethanone (15.1 g, 0.08 mol) in EtOH (156 mL) at 0° C. under N₂ atmosphere were added Et₃N (10.7 mL, 0.076 mol) and hydrazine hydrate (4 mL, 0.08 mol). The reaction mixture was stirred for 2 h at RT. After consumption of the starting material (monitored by TLC), the volatiles from the reaction were removed under reduced pressure. The residue was diluted with water and extracted with EtOAc. The combined organic layers were dried over anhydrous Na₂SO₄, filtered and concentrated under vacuum. Obtained crude material was diluted with ether and further stirred for 10 min, filtered and filtrate was concentrated under reduced pressure to afford 4-chloro-3-methyl-1H-pyrazolo [3, 4-d] pyrimidine (12.6 g, 94%) as yellow solid.

TLC: 20% EtOAc/Hexane (R_{f}: 0.2)

### Example 1: 2-[4-(3-bromo-1H-pyrazolo[3,4-d]pyrimidin-4-yl)piperazin-1-yl]-2-(4-chlorophenyl)-N,N-dimethyl-ethanamine

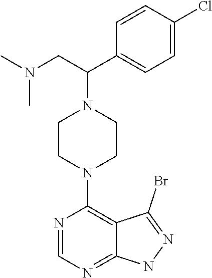 [SMILES: CN(C)CC(c1ccc(Cl)cc1)N1CCN(c2ncnc3[nH]nc(Br)c23)CC1]

To a stirred solution of Intermediate 6 (335 mg, 1.05 mmol) in THF (10 mL) was added 3-bromo-4-chloro-1H-pyrazolo[3,4-d]pyrimidine (293 mg, 1.05 mmol) followed by Et₃N (0.88 mL, 6.32 mmol) at 0° C. under N₂ atmosphere. The resulting reaction mixture was heated to 80° C. and stirred for 2 h. After consumption of the starting material (monitored by TLC), the reaction mixture was diluted with EtOAc and washed with water. The organic layer was dried over anhydrous Na₂SO₄, filtered and concentrated under reduced pressure. The obtained crude material was purified by silica gel column chromatography eluting with 4% MeOH/CH₂Cl₂ to afford Example 1 (500 mg, 90%) as an off-white solid.

TLC: 5% MeOH/DCM (R_{f}: 0.4)

¹HNMR (500 MHz, CD₃OD): δ 8.22 (s, 1H), 7.37 (d, J=8.5 Hz, 2H), 7.29 (d, J=8.0 Hz, 2H), 3.90-3.85 (m, 5H), 3.19-3.15 (m, 1H), 2.84-2.80 (m, 1H), 2.67-2.62 (m, 2H), 2.60-2.55 (m, 2H), 2.41 (s, 6H).

LC-MS: m/z 466 [M+1]⁺ at 5.08 RT (95.54% purity) (Column: Ascentis (R) Express C18 (150×4.6 mm, 2.72μ); λ=218 nm

HPLC purity: 97.07%

Example compounds 3a, 3b, 4, 5, 7, 9, 11, 14 and 15 which are shown in Table 1, below, may be made using commercially available starting materials, Intermediate 8, Intermediate 9, or Intermediate 10, following analogous procedures to General Method I.

TABLE 1

| Ex. No | Structure | Name |
|---|---|---|
| 4 | 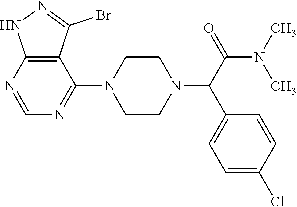 [SMILES: CN(C)C(=O)C(c1ccc(Cl)cc1)N1CCN(c2ncnc3[nH]nc(Br)c23)CC1] | 2-(4-(3-bromo-1H-pyrazolo[3,4- d]pyrimidin-4-yl)piperazin-1-yl)-2-(4- chlorophenyl)-N,N-dimethylacetamide |
| 5 | 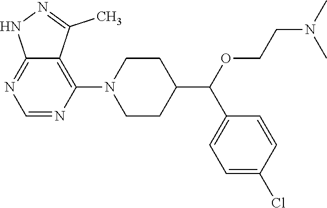 [SMILES: Cc1n[nH]c2ncnc(N3CCC(C(OCCN(C)C)c4ccc(Cl)cc4)CC3)c12] | 2-((4-chlorophenyl)(1-(3-methyl-1H- pyrazolo[3,4-d]pyrimidin-4-yl)piperidin- 4-yl)methoxy)-N,N-dimethylethan-1- amine |
| 7 | 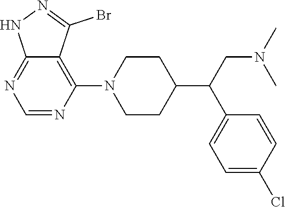 [SMILES: CN(C)CC(c1ccc(Cl)cc1)C1CCN(c2ncnc3[nH]nc(Br)c23)CC1] | 2-(1-(3-bromo-1H-pyrazolo[3,4- d]pyrimidin-4-yl)piperidin-4-yl)-2-(4- chlorophenyl)-N,N-dimethylethan-1- amine |
| 3a | 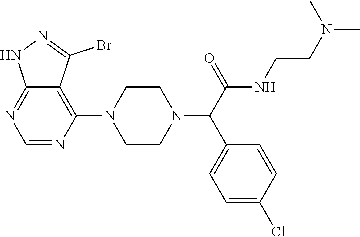 [SMILES: CN(C)CCNC(=O)C(c1ccc(Cl)cc1)N1CCN(c2ncnc3[nH]nc(Br)c23)CC1] | (−)-2-(4-(3-bromo-1H-pyrazolo[3,4- d]pyrimidin-4-yl)piperazin-1-yl)-2-(4- chlorophenyl)-N-(2- (dimethylamino)ethyl)acetamide |
| 9 | 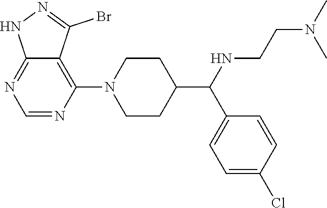 [SMILES: CN(C)CCNC(c1ccc(Cl)cc1)C1CCN(c2ncnc3[nH]nc(Br)c23)CC1] | N¹-((1-(3-bromo-1H-pyrazolo[3,4- d]pyrimidin-4-yl)piperidin-4-yl)(4- chlorophenyl)methyl)-N²,N²- dimethylethane-1,2-diamine |
| 3b | 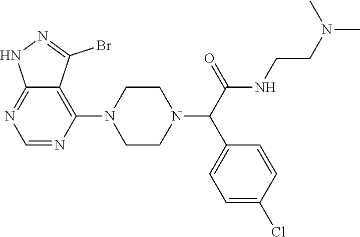 [SMILES: CN(C)CCNC(=O)C(c1ccc(Cl)cc1)N1CCN(c2ncnc3[nH]nc(Br)c23)CC1] | (+)-2-(4-(3-bromo-1H-pyrazolo[3,4- d]pyrimidin-4-yl)piperazin-1-yl)-2-(4- chlorophenyl)-N-(2- (dimethylamino)ethyl)acetamide |
| 11 | 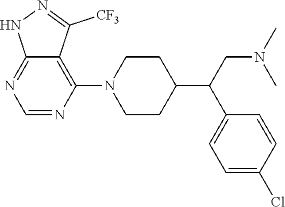 [SMILES: CN(C)CC(c1ccc(Cl)cc1)C1CCN(c2ncnc3[nH]nc(C(F)(F)F)c23)CC1] | 2-(4-chlorophenyl)-N,N-dimethyl-2-(1- (3-(trifluoromethyl)-1H-pyrazolo[3,4- d]pyrimidin-4-yl)piperidin-4-yl)ethan-1- amine |
| 14 | 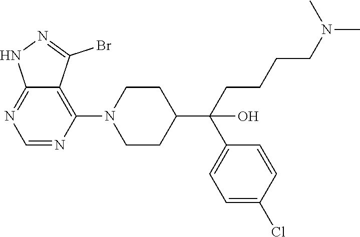 [SMILES: CN(C)CCCCC(O)(c1ccc(Cl)cc1)C1CCN(c2ncnc3[nH]nc(Br)c23)CC1] | 1-(1-(3-bromo-1H-pyrazolo[3,4- d]pyrimidin-4-yl)piperidin-4-yl)-1-(4- chlorophenyl)-5- (dimethylamino)pentan-1-ol |
| 15 | 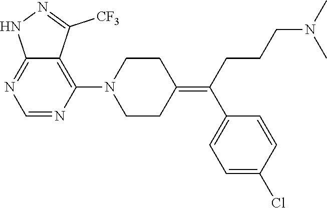 [SMILES: CN(C)CCCC(=C1CCN(c2ncnc3[nH]nc(C(F)(F)F)c23)CC1)c1ccc(Cl)cc1] | 4-(4-chlorophenyl)-N,N-dimethyl-4-(1- (3-(trifluoromethyl)-1H-pyrazolo[3,4- d]pyrimidin-4-yl)piperidin-4- ylidene)butan-1-amine |

### Synthesis of Example Compounds 12a, 12b and 13

### Intermediate 9: (1-(1-(tert-butyl)-3-(trifluoromethyl)-1H-pyrazalo[3,4-d]pyrimidin-4-yl)piperidin-4-yl)(4-chlorophenyl) methanone

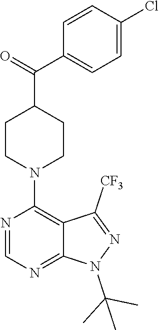 [SMILES: CC(C)(C)n1nc(C(F)(F)F)c2c(N3CCC(C(=O)c4ccc(Cl)cc4)CC3)ncnc21]

To a stirred solution of (4-chlorophenyl)(piperidin-4-yl)methanone (200 mg, 0.89 mmol) in THF (5 mL) was added Intermediate 7 (274 mg, 0.98 mmol) followed by Et₃N (0.747 mL, 5.38 mmol) at 0° C. under N₂ atmosphere. The resulting reaction mixture was heated to 80° C. and stirred for 2 h. After consumption of the starting material (monitored by TLC), the reaction was diluted with EtOAc and washed with water. The separated organic layer was dried over anhydrous Na₂SO₄, filtered and concentrated under reduced pressure. The obtained crude material was purified by silica gel column chromatography eluting with 15% EtOAc/Hexane to afford Intermediate 9 (390 mg, 94%) as pale-yellow solid.

TLC: 20% EtOAc/Hexane (R_{f}: 0.8)

¹HNMR (500 MHz, CDCl₃): δ 8.42 (s, 1H), 7.94 (d, 2H), 7.48 (d, 2H), 4.36-4.32 (m, 2H), 3.54-3.52 (m, 1H), 3.31-3.27 (m, 2H), 1.99-1.95 (m, 4H), 1.82 (s, 9H)

LC-MS: m/z 466.6 [M+1]⁺ at 5.13 RT (83.02% purity)

### Intermediate 10: (4-chlorophenyl)(1-(3-(trifluoromethyl)-1H-pyrazolo[3,4-d]pyrimidin-4-yl)piperidin-4-yl)methanone

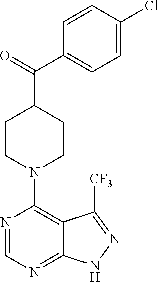 [SMILES: O=C(c1ccc(Cl)cc1)C1CCN(c2ncnc3[nH]nc(C(F)(F)F)c23)CC1]

To Intermediate 9 (400 mg, 0.85 mmol), under N₂ atmosphere, was added methanesulfonic acid (4 mL) at 0° C. and the reaction mixture was stirred at RT for 16 h. The resulting reaction mixture was poured into ice cold water, basified with saturated NaHCO₃ solution and extracted with EtOAc. The organic layer was dried over anhydrous Na₂SO₄, filtered and concentrated under reduced pressure. The obtained crude material was purified by silica gel column chromatography eluting with 40% EtOAc/Hexane to afford Intermediate 10 (300 mg, 85%) as white solid.

TLC: 40% EtOAc/Hexane (R_{f}: 0.8)

### ¹HNMR (500 MHz, DMSO-ds): δ 14.57 (bs, 1H), 8.42 (s, 1H), 8.03 (d, 2H), 7.61 (d, 2H), 4.24-4.21 (m, 4H), 3.82-3.80 (m, 1H), 1.94-1.92 (m, 2H), 1.69-1.64 (m, 2H)

LC-MS: m/z 410 [M+1]⁺ at 3.94 RT (99.36% purity)

### Example 12a and 12b: N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine (BCF₃-E1(H)—NHC₂NMe₂

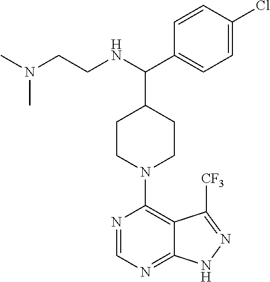 [SMILES: CN(C)CCNC(c1ccc(Cl)cc1)C1CCN(c2ncnc3[nH]nc(C(F)(F)F)c23)CC1]

To Intermediate 10, (125 mg, 0.30 mmol) was added N¹,N¹-dimethylethane-1,2-diamine (0.06 mL, 0.61 mmol) and Ti(Oipr)₄ (0.363 mL, 1.22 mmol) at 0° C. under inert atmosphere. After being stirred for 12 h at RT, MeOH (1.25 mL), AcOH (0.412 mL) and NaBH₄ (23 mg, 0.60 mmol) at 0° C. was added to the above solution and continued stirring for further 1 h at RT; progress of the reaction was monitored by TLC. The reaction mixture was filtered through a celite bed and washed with EtOAc. The obtained filtrate was washed with 5% NaOH, brine dried over anhydrous Na₂SO₄ and concentrated under reduced pressure to obtain the crude. The crude material was purified by Chiral preparative HPLC (described in further detail below) to afford Example 12a (110 mg, 46%) and Example 12b (110 mg, 46%) both as white solids.

Example 12a is (+)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine. Example 12b is (−)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine.

Characterising data for Example 12a and Example 12b are shown in Table 2 below.

### Intermediate 11: 2-(4-chlorophenyl)-N,N-dimethyl-2-(piperazin-1-yl)acetamide hydrochloride

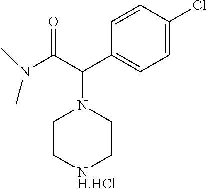

To a stirred solution of Intermediate 4, (400 mg, 1.05 mmol) in MeOH (3.2 mL) was added 4N HCl in 1,4-Dioxane (3.2 mL) at 0° C. under inert atmosphere. The resulting reaction mixture was allowed to warm to RT and stirred for 2 h; progress of the reaction was monitored by TLC. The reaction mixture was concentrated under reduced pressure to afford Intermediate 11 (335 mg, crude) as an off-white solid. This material was directly used fto make intermediate 12 without further purification.

TLC: 5% MeOH/CH₂Cl₂ (R_{f}: 0.15)

LC-MS: m/z 282 [M+1]⁺ at 2.30 RT (89.59% purity) (Column: X-bridge C-18 (50×3.0 mm x 3.5μ); 5 mM NH₄Oac in water: ACN; 0.8 ml/min)

### Intermediate 12: 2-(4-(1-(tert-butyl)-3-(trifluoromethyl)-1H-pyrazolo[3,4-d]pyrimidin-4-yl)piperazin-1-yl)-2-(4-chlorophenyl)-N,N-dimethylacetamide

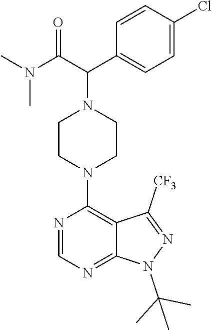 [SMILES: CN(C)C(=O)C(c1ccc(Cl)cc1)N1CCN(c2ncnc3c2c(C(F)(F)F)nn3C(C)(C)C)CC1]

To a stirred solution of Intermediate 11 (335 mg, 1.05 mmol) in THF (10 mL) was added Intermediate 7 (293 mg, 1.05 mmol) followed by Et₃N (0.88 mL, 6.32 mmol) at 0° C. under N₂ atmosphere. The resulting reaction mixture was heated to 80° C. and stirred for 2 h. After consumption of the starting material (monitored by TLC), the reaction mixture was diluted with EtOAc and washed with water. The organic layer was dried over anhydrous Na₂SO₄, filtered and concentrated under reduced pressure. The obtained crude material was purified by silica gel column chromatography eluting with 4% MeOH/CH₂Cl₂ to afford Intermediate 12 (500 mg, 90%) as an off-white solid.

TLC: 10% EtOAc/Hexane (R_{f}: 0.2)

¹HNMR (400 MHz, CDCl₃): δ 8.38 (s, 1H), 7.41-7.34 (m, 4H), 4.31 (s, 1H), 3.76-3.71 (m, 4H), 2.98 (s, 3H), 2.96 (s, 3H), 2.63-2.59 (m, 4H), 1.82 (s, 9H)

LC-MS: m/z 524.2 [M+1]⁺ at 5.04 RT (94.57% purity) (Column: X-bridge C-18 (50×3.0 mm×3.5μ); 5 mM NH₄OAc: ACN; 0.8 ml/min)

### Intermediate 13: 2-(4-chlorophenyl)-N,N-dimethyl-2-(4-(3-(trifluoromethyl)-1H-pyrazolo[3,4-d]pyrimidin-4-yl)piperazin-1-yl)

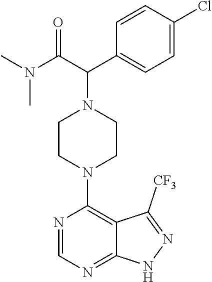 [SMILES: CN(C)C(=O)C(c1ccc(Cl)cc1)N1CCN(c2ncnc3[nH]nc(C(F)(F)F)c23)CC1]

To Intermediate 12 (500 mg, 0.95 mmol), under N₂ atmosphere, was added methanesulfonic acid (5 mL) at 0° C. and the reaction mixture was stirred at RT for 16 h. The resulting reaction mixture was poured into ice cold water, basified with saturated NaHCO₃ and extracted with EtOAc. The combined organic extracts were dried over anhydrous Na₂SO₄, filtered and concentrated under reduced pressure. Obtained crude material was purified by silica gel column chromatography eluting with 3% MeOH/CH₂Cl₂ to afford Intermediate 13 (350 mg, 78%) as an off-white solid.

TLC: 10% MeOH/CH₂Cl₂ (R_{f}: 0.7)

¹HNMR (500 MHz, CD₃OD): δ8.33 (s, 1H), 7.47 (d, J=8.0 Hz, 2H), 7.28 (d, J=8.0 Hz, 2H), 4.48 (s, 1H), 3.78-3.77 (m, 4H), 3.03 (s, 3H), 2.92 (s, 3H), 2.61-2.53 (m, 4H)

LC-MS: m/z 468 [M+1]⁺ at 3.38 RT (99.59% purity) (Column: X-bridge C-18 (150×4.6 mm x 3.5μ); 5 mM NH₄OAc: ACN; 0.8 ml/min); λ=216 nm

### Example 13: 2-(4-chlorophenyl)-N,N-dimethyl-2-(4-(3-(trifluoromethyl)-1H-pyrazolo[3,4-d]pyrimidin-4-yl)piperazin-1-yl)ethanamine

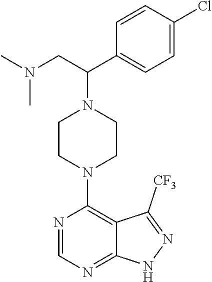 [SMILES: CN(C)CC(c1ccc(Cl)cc1)N1CCN(c2ncnc3[nH]nc(C(F)(F)F)c23)CC1]

To a stirred solution Intermediate 13, (200 mg, 0.42 mmol) in dry THF (5 mL) was added LAH (0.17 mL, 0.34 mmol; 2M in THF) at 0° C. and stirred for 1 h under N₂ atmosphere. After consumption of the starting material (monitored by TLC), the reaction mass was poured into saturated NH₄Cl and extracted with EtOAc. The combined organic extracts were dried over anhydrous Na₂SO₄, filtered and concentrated under reduced pressure to get the crude material; which was purified by Chiral preparative HPLC to afford Example 13 (80 mg, 41%) as an off-white solid.

Characterising data for Example 13 is shown in Table 2 below.

Characterising data for Example compounds 3a, 3b, 4, 5, 7, 9, 11, 12a, 12b, 13, 14 and 15 are shown in Table 2 below. For Example compounds 3a and 3b, and compounds 12a and 12b, an additional step of purification of the racemic mixture was carried out by chiral preparative HPLC. The racemic mixture of compounds 3a and 3b was purified by Chiralpak IC, 250×4.6 mm, 5 m (0.1% TEA in n-Hexane: ethanol (50:50) as mobile phase) to obtain fraction 1 (non-polar peak) and fraction 2 (polar peak). Fraction 1 was the dextrorotary (+) isomer and is Compound 3b; fraction 2 was the levorotary (−) isomer and is Compound 3a. The racemic mixture of compounds 12a and 12b was purified by Chiralpak IC, 250×4.6 mm, 5 μm (mobile phase (A) 0.1% TEA in n-Hexane (B) DCM:MeOH (80:20) (A:B:85:15); flow Rate: 1.00 mL/min) to obtain fraction 1 (non-polar peak) and fraction 2 (polar peak). Fraction 1 was the dextrorotary (+) isomer and is Compound 12a; fraction 2 was the levorotary (−) isomer and is Compound 12b.

TABLE 2

| | | LCMS and/or HPLC and/or |
|---|---|---|
| Ex. | | melting range and/or Optical |
| No | NMR δ_{H}/ppm | Rotation results |
| 4 | ¹HNMR (500 MHz, CDCl3): δ 12.81 (bs, 1H), 8.36 (s, | LC-MS: m/z 480 [M + 2]⁺ at |
| | 1H), 7.37 (d, J = 8.5 Hz, 4H), 4.35 (s, 1H), 3.97 (t, J = | 4.34 RT (98.64% purity) |
| | 5.0 Hz, 4H), 2.97 (d, J = 11.0 Hz, 6H), 2.75-2.65 (m, | (Column: Ascentis (R) Express |
| | 4H) | C18 (150 × 4.6 mm, 2.7 μ); λ = |
| | | 220 nm |
| 5 | ¹HNMR (500 MHz, CDCl₃): δ 11.70 (bs, 1H), 8.38 (s, | Mass: m/z 429 [M + 1]⁺ |
| | 1H), 7.35-7.23 (m, 4H), 4.45 (d, J = 13.5 Hz, 1H), 4.36 | HPLC: 97.79% |
| | (d, J = 13.5 Hz, 1H), 3.94 (d, J = 8.0 Hz, 1H), 3.43-3.39 | Melting Range: 222.8° C. to |
| | (m, 2H), 3.07-2.92 (m, 2H), 2.61 (s, 3H), 2.55-2.44 | 224.2° C. |
| | (m, 2H), 2.24 (s, 7H), 1.94-1.88 (m, 1H), 1.49-1.30 | |
| | (m, 3H) | |
| 7 | ¹HNMR (500 MHz, DMSO-d₆): δ 13.98-13.96 (bs, | LC-MS: m/z 465 [M + 1]⁺ at |
| | 1H), 8.25 (s, 1H), 7.38 (d, J = 8 Hz, 2H), 7.31 (d, J = 8 | 12.54 RT (94.39% purity) |
| | Hz, 2H), 4.47 (d, J = 12.5 Hz, 1H), 4.37 (d,, J = 12.5 | (Column: X-Terra RP-8 C-18 |
| | Hz, 1H), 3.34-3.32 (m, 1H), 3.08-3.03 (m, 1H), 2.98- | (250 × 4.6 mm × 5.0 μ); 0.01% |
| | 2.94 (m, 2H), 2.00 (s, 6H), 1.98-1.96 (m, 2H), 1.90- | TFA: ACN; 1.0 ml/min); λ = |
| | 1.86 (m, 1H), 1.38-1.33 (m, 2H), 1.17-1.14 (m, 1H) | 220 nm |
| 3a | ¹HNMR (500 MHz, CDCl₃): δ 8.38 (s, 1H), 7.54 (bs, | Chiral LC-MS: 522.9 [M + 2] |
| | 1H), 7.32 (s, 4H), 3.86-3.82 (m, 5H), 3.41 (q, J = 5.5 | HPLC: 99.27% |
| | Hz, 2H), 2.58-2.48 (m, 6H), 2.31 (s, 6H). | Chiral HPLC Purity: 99.79%. |
| | | Optical rotation [α]_{D}²⁰: |
| | | −91.59°, (c = 1% in DCM) |
| 9 | ¹HNMR (500 MHz, DMSO-d₆): δ 8.26 (s, 1H), 7.38 | Mass: m/z 494 [M + 1]⁺ |
| | (d,, J = 8.5 Hz, 2H), 7.32 (d,, J = 8.5 Hz, 2H), 4.49 (d, | HPLC: 97.65% |
| | J = 13 Hz, 1H), 4.38 (d, J = 13 Hz, 1H), 3.40-3.37 (m, | |
| | 2H), 3.04 (t, J = 12.5 Hz, 1H), 2.95 (t, J = 12.5 Hz, | |
| | 1H), 2.35-2.33 (m, 4H), 2.11 (s, 7H), 1.82-1.78 (m, | |
| | 1H), 1.34-1.21 (m, 3H) | |
| 3b | ¹HNMR (500 MHz, CDCl₃): δ: 8.38 (s, 1H), 7.52 (bs, | MS: m/e: 522.9 (M + 2) |
| | 1H), 7.32 (s, 4H), 3.86-3.83 (m, 5H), 3.40 (q, J = 5.0 | HPLC: 96.95% |
| | Hz, 2H), 2.58-2.56 (m, 6H), 2.30 (s, 6H) | Chiral HPLC Purity: 99.87%. |
| | | Optical rotation [α]_{D}²⁰:: + |
| | | 86.92° (c = 1% in DCM) |
| 11 | ¹HNMR (400 MHz, CD₃OD): δ 8.31 (s, 1H), 7.32 (d, | LC-MS: m/z 453.3 [M + 1]⁺ at |
| | J = 8.4 Hz, 2H), 7.22 (d, J = 8.4 Hz, 2H), 4.39 (d, J = | 3.19 RT (97.68% purity) |
| | 13.6 Hz, 1H), 7.29 (d, J = 13.2 Hz, 1H), 3.17-3.10 (m, | (Column: X-select C-18 (50 × |
| | 1H), 3.06-2.99 (m, 1H), 2.87-2.82 (m, 1H), 2.78-2.71 | 3.0 mm × 3.5 μ); 5 mM NH₄OAc: |
| | (m, 2H), 2.21 (s, 6H), 2.07-1.99 (m, 1H), 1.89-1.85 | ACN; 0.8 ml/min); λ = 226 nm |
| | (m, 1H), 1.41-1.32 (m, 1H), 1.27-1.24 (m, 1H), 1.22- | |
| | 1.18 (m, 1H) | |
| 12a | ¹HNMR (400 MHz, CD₃OD): δ 8.30 (s, 1H), 7.33-7.30 | LC-MS: m/z 482.4 [M + 1]⁺ at |
| | (m, 4H), 4.39 (d, J = 14.0 Hz, 1H), 4.28 (d, J = 13.6 Hz, | 3.37 RT (97.88% purity) |
| | 1H), 3.40 (d, J = 7.6 Hz, 1H), 3.16-3.09 (m, 1H), 3.04- | (Column: X-select C-18 (50 × |
| | 2.97 (m, 1H), 2.49-2.42 (m, 4H), 2.26 (s, 6H), 2.15- | 3.0 mm × 3.5 μ); 5 mM NH₄OAc: |
| | 2.11 (m, 1H), 1.94-1.86 (m, 1H), 1.43-1.19 (m, 4H) | ACN; 0.8 ml/min); λ = 220 nm |
| | | Chiral HPLC: 100.00% ee Rₜ = |
| | | 9.25 min |
| | | Optical rotation [α]_{D}²⁰: |
| | | +43.30° (c = 0.5% in MeOH) |
| 13 | ¹HNMR (400 MHz, CD₃OD): δ 8.31 (s, 1H), 7.37 (d, | LC-MS: m/z 454 [M + 1]⁺ at |
| | J = 8.4 Hz, 2H), 7.28 (d, J = 8.4 Hz, 2H), 3.84-3.77 | 2.98 RT (99.38% purity) |
| | (m, 5H), 3.10-3.05 (m, 1H), 2.80-2.75 (m, 1H), | (Column: X-bridge C-18 (50 × |
| | 2.61-2.51 (m, 4H), 2.32 (s, 6H) | 3.0 mm × 3.5 μ); 5mM NH₄OAc: |
| | | ACN; 0.8 ml/min); λ = 228 nm |
| 14 | ¹HNMR (500 MHz, DMSO-d₆): δ 13.96 (bs, 1H), 9.23 | Mass: m/z 523 [M + 1] |
| | (bs, 1H), 8.26 (s, 1H), 7.41-7.35 (m, 4H), 4.56 (d, J = | HPLC: 99.49% |
| | 13 Hz, 1H), 4.42 (d, J = 13 Hz, 1H), 3.04-2.99 (m, 1H), | |
| | 2.91-2.89 (m, 3H), 2.70 (s, 6H), 1.94-1.79 (m, 4H), | |
| | 1.52-1.43 (m, 4H), 1.39-1.37 (m, 2H), 0.77-0.75 (m, | |
| | 1H) | |
| 15 | ¹HNMR (400 MHz, CD₃OD): δ 8.34 (s, 1H), 7.35 (d, | LC-MS: m/z 479 [M + 1]⁺ at |
| | J = 8.4 Hz, 2H), 7.13 (d, J = 8.4 Hz, 2H), 3.85 (t, J = 6.0 | 3.36 (99.72% purity) (Column: |
| | Hz, 2H), 3.68 (t, J = 6.0 Hz, 2H), 2.61 (t, J = 6.0 Hz, | X-bridge C-18 (150 × 4.6 mm × |
| | 2H), 2.46 (t, J = 7.6 Hz, 2H), 2.35 (t, J = 8.0 Hz, 2H), | 3.5 μ); 5 mM NH₄OAc: ACN; |
| | 2.25-2.24 (m, 2H), 2.22 (s, 6H), 1.51-1.44 (m, 2H) | 0.8 ml/min); λ = 221 nm |
| 12b | ¹HNMR (400 MHz, CD₃OD): δ 8.30 (s, 1H), 7.35-7.28 | LC-MS: m/z 482.4 [M + 1]⁺ at |
| | (m, 4H), 4.39 (d, J = 13.6 Hz, 1H), 4.28 (d, J = 13.2 Hz, | 3.37 RT (97.85% purity) |
| | 1H), 3.40 (d, J = 8.0 Hz, 1H), 3.16-3.09 (m, 1H), 3.04- | (Column: X-select C-18 (50 × |
| | 2.97 (m, 1H), 2.50-2.43 (m, 4H), 2.24 (s, 6H), 2.15- | 3.0 mm x 3.5 μ); 5 mM NH₄OAc: |
| | 2.11 (m, 1H), 1.95-1.86 (m, 1H), 1.43-1.16 (m, 4H) | ACN; 0.8 ml/min); λ = 220 nm |
| | | Chiral HPLC: 100.00% ee Rₜ = |
| | | 15.53 min |
| | | Optical rotation [α]_{D}²⁰ |
| | | −39.73° (c = 0.5 % in MeOH) |

Examples 2, 3, 6, 8 and 10 may be synthesised following the procedure disclosed in US2008/0188482:

TABLE 3

| | | | Example number |
|---|---|---|---|
| Ex | | | in |
| No. | Structure | Name | US2008/018848 |
| 2 | 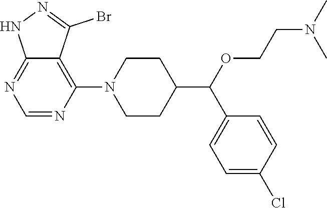 [SMILES: CN(C)CCOC(c1ccc(Cl)cc1)C1CCN(c2ncnc3[nH]nc(Br)c23)CC1] | 2-((1-(3-bromo-1H- pyrazolo[3,4-d]pyrimidin- 4-yl)piperidin-4-yl)(4- chlorophenyl)methoxy)- N,N-dimethylethan-1- amine | 2 |
| 3 | 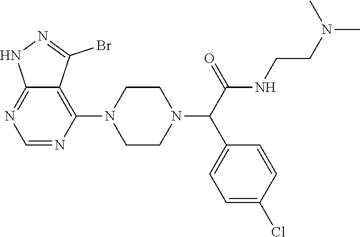 [SMILES: CN(C)CCNC(=O)C(c1ccc(Cl)cc1)N1CCN(c2ncnc3[nH]nc(Br)c23)CC1] | 2-(4-(3-bromo-1H- pyrazolo[3,4-d]pyrimidin- 4-yl)piperazin-1-yl)-2-(4- chlorophenyl)-N-(2- (dimethylamino)ethyl)acet amide | 9 |
| 6 | 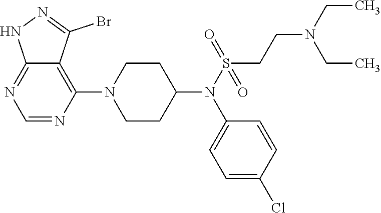 [SMILES: CCN(CC)CCS(=O)(=O)N(c1ccc(Cl)cc1)C1CCN(c2ncnc3[nH]nc(Br)c23)CC1] | N-(1-(3-bromo-1H- pyrazolo[3,4-d]pyrimidin- 4-yl)piperidin-4-yl)-N-(4- chlorophenyl)-2- (diethylamino)ethane-1- sulfonamide | 29 |
| 8 | 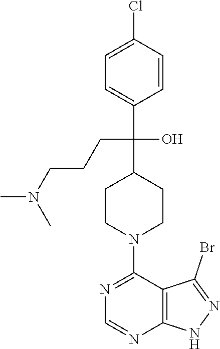 [SMILES: CN(C)CCCC(O)(c1ccc(Cl)cc1)C1CCN(c2ncnc3[nH]nc(Br)c23)CC1] | 1-(1-(3-bromo-1H- pyrazolo[3,4-d]pyrimidin- 4-yl)piperidin-4-yl)-1-(4- chlorophenyl)-4- (dimethylamino)butan-1-ol | 91 |
| 10 | 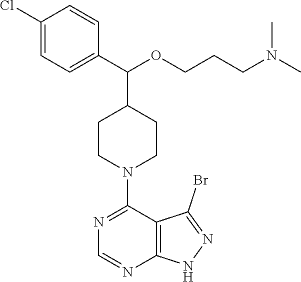 [SMILES: CN(C)CCCOC(c1ccc(Cl)cc1)C1CCN(c2ncnc3[nH]nc(Br)c23)CC1] | 3-((1-(3-bromo-1H- pyrazolo[3,4-d]pyrimidin- 4-yl)piperidin-4-yl)(4- chlorophenyl)methoxy)- N,N-dimethylpropan-1- amine | 3 |

### Enzymatic Inhibition Assays

### Description of the Enzymatic Inhibition Assays:

### AKT1, AKT2, AKT3

The IC₅₀ values of Example compounds of the invention for AKT1, AKT2 and AKT3 were measured using HTRF (Homogeneous Time Resolved Fluorescence) KinEASE assays (62ST3PEB—1000 tests or 62ST3PEC—20,000 tests) from cisbio.com, according to manufacturer's instructions.

### DNA-PK

The IC₅₀ values of Example compounds of the invention for DNA-PK were measured using the HTRF KinEASE assay as described above.

The Reference compound used in the AKT1, AKT2, AKT3 and DNA-PK enzymatic inhibition assays was triciribine in its phosphate salt form (((2R,3S,4R,5R)-5-(3-amino-5-methyl-1,4,5,6,8-pentaazaacenaphthylen-1(5H)-yl)-3,4-dihydroxytetrahydrofuran-2-yl)methyl dihydrogen phosphate, Sigma Aldrich, UK), a known AKT inhibitor. Triciribine is a PH domain inhibitor and does not bind to the AKT catalytic domain. Triciribine is inactive in the AKT enzyme assay, which does not contain full length protein.

The structure of triciribine phosphate is as follows:

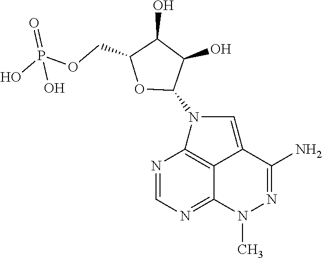 [SMILES: CN1N=C(N)c2cn([C@@H]3O[C@H](COP(=O)(O)O)[C@@H](O)[C@H]3O)c3ncnc1c23]

Roche comp-4 (synthesised following the procedure disclosed in Breitenlechner C B, Journal of Medicinal Chemistry, 2004, 47, issue 6, pages 1375-1390), a known AKT inhibitor, was also tested in this assay as a comparative example. Roche comp-4 is N-{(3R,4R)-4-[4-(2-fluoro-6-hydroxy-3-methoxy-benzoyl)-benzoylamino]-azepan-3-yl}-isonicotinamide. It is disclosed as Example 4 in Breitenlechner C B, Journal of Medicinal Chemistry, 2004, 47, issue 6, pages 1375-1390, and its structure is as follows:

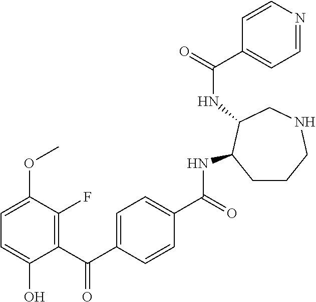 [SMILES: COc1ccc(O)c(C(=O)c2ccc(C(=O)N[C@@H]3CCCNC[C@H]3NC(=O)c3ccncc3)cc2)c1F]

GSK-3g (GSK-690693, synthesised following the procedure disclosed in Heerding, D. A., et al, Journal of Medicinal Chemistry, 2008, Vol. 51, pages 5663-5679) a known AKT inhibitor, was also tested in this assay as a comparative example. GSK-3g is 4-[2-(4-amino-1,2,5-oxadiazol-3-yl)-1-ethyl-7-[[(3S)-piperidin-3-yl]methoxy]imidazo[4,5-c]pyridin-4-yl]-2-methylbut-3-yn-2-ol. It is disclosed as Example 3g in Heerding, D. A., et al, Journal of Medicinal Chemistry, 2008, Vol. 51, pages 5663-5679, and its structure is as follows:

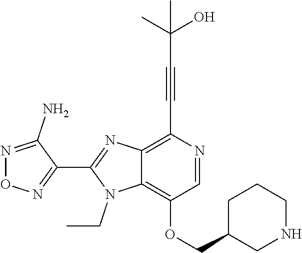 [SMILES: CCn1c(-c2nonc2N)nc2c(C#CC(C)(C)O)ncc(OC[C@H]3CCCNC3)c21]

### Results

The results of the AKT1, AKT2, AKT3 and DNA-PK enzymatic inhibition assays are shown in Table 4 below. As can be seen from the results in Table 4, all compounds of the invention are dual AKT and DNA-PK inhibitors as all compound have a pIC₅₀ of 6 or greater for at least one of AKT1, AKT2 and/or AKT3, and a pIC₅₀ of 6 or greater for DNA-PK. Furthermore, the majority of compounds listed have a pIC₅₀ of 7.5 or greater or 8.0 or greater for each of AKT1, AKT2 and AKT3, and have a pIC₅₀ of 7.5 or greater or 8.0 or greater for DNA-PK. Example 1 demonstrates potency of inhibition for each AKT isoform and DNA-PK with pIC50 of 9 or greater for each enzyme target.

TABLE 4

| Example | AKT1 | AKT2 | AKT3 | DNA- |
|---|---|---|---|---|
| No. | pIC₅₀ | pIC₅₀ | pIC₅₀ | PK pIC₅₀ |
| 1 | 9.49 | 9.26 | 9.95 | 9.62 |
| 2 | 8.24 | 7.98 | 8.44 | 8.41 |
| 3a | 8.8 | 8.44 | 8.96 | 7.97 |
| 4 | 5.87 | 5.65 | 6.22 | 6.85 |
| 5 | 7.9 | 7.56 | 8.14 | 8.15 |
| 6 | 8.88 | 8.13 | 9.07 | 8.21 |
| 7 | 9.04 | 8.44 | 8.86 | 8.56 |
| 8 | 8.59 | 8.22 | 9.02 | 8.46 |
| 3a | 8.35 | 7.88 | 8.44 | 8.31 |
| 9 | 8.92 | 8.56 | 9.17 | 8.71 |
| 3b | 7.82 | 7.46 | 8.28 | 7.98 |
| 11 | 9.04 | 8.78 | 9.5 | 8.77 |
| 12a | 9.24 | 8.85 | 9.66 | 9.8 |
| 13 | 8.15 | 8.14 | 8.53 | 8.73 |
| 14 | 8.6 | 8.19 | 8.89 | 8.33 |
| 15 | 6.03 | 6.31 | 6.80 | 7.92 |
| 12b | 8.47 | 8.26 | 9.08 | 8.27 |
| Roche comp-4 | 6.03 | 6.77 | 6.68 | 8.07 |
| GSK-3g | 9.15 | 9.66 | 8.6 | 7.98 |
| (GSK-690693) | | | | |

### In Vitro Cell-Based Phenotypic Assays

Apoptosis was assessed using Caspase-Glo® 3/7 (Promega, US), described in detail below. according to manufacturer's instructions. The apoptosis caspase assay was used for different experiments/assays as outlined below, the basic protocol for assay read-out remained the same for each different experiment.

Cell viability was assessed using MTT assay, described in detail below, using standard protocols. The MTT assay was used for different experiments/assays as outlined below, however the basic protocol for assessing MTT read-out in each experiment following different experimental set up remained the same.

Cell lines used were: the isogenic paired high grade serous ovarian cancer platinum sensitive PEO1 cell line (obtained from Dr Simon Langdon, Edinburgh University); the isogenic paired high grade serous ovarian cancer platinum sensitive platinum resistant PEO4 cell line (obtained from Dr Simon Langdon, Edinburgh University); and the platinum resistant epithelial ovarian cancer cell line SKOV3 (obtained from ATCC).

The comparative example compounds Afuresertib, Uprosertib, Ipatasertib, triciribine, NU7441, and LY3023414 were obtained from Selleck Chem, UK.

Afuresertib is N-[(2S)-1-amino-3-(3-fluorophenyl)propan-2-yl]-5-chloro-4-(4-chloro-2-methylpyrazol-3-yl)thiophene-2-carboxamide. It is a known AKT inhibitor, and has the following structure:

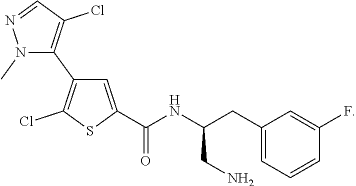 [SMILES: Cn1ncc(Cl)c1-c1cc(C(=O)N[C@H](CN)Cc2cccc(F)c2)sc1Cl]

Uprosertib is N-[(2S)-1-amino-3-(3,4-difluorophenyl)propan-2-yl]-5-chloro-4-(4-chloro-2-methylpyrazol-3-yl)furan-2-carboxamide. It is a known AKT inhibitor and has the following structure:

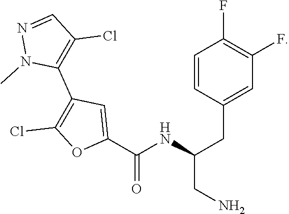 [SMILES: Cn1ncc(Cl)c1-c1cc(C(=O)N[C@H](CN)Cc2ccc(F)c(F)c2)oc1Cl]

Ipatasertib is (2S)-2-(4-chlorophenyl)-1-[4-[(5R,7R)-7-hydroxy-5-methyl-6,7-dihydro-5H-cyclopenta[d]pyrimidin-4-yl]piperazin-1-yl]-3-(propan-2-ylamino)propan-1-one. It is a known AKT inhibitor and has the following structure:

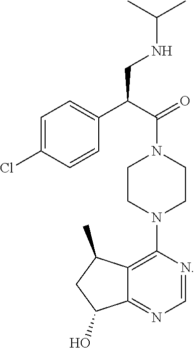 [SMILES: CC(C)NC[C@@H](C(=O)N1CCN(c2ncnc3c2[C@H](C)C[C@H]3O)CC1)c1ccc(Cl)cc1]

NU7441 is 8-dibenzothiophen-4-yl-2-morpholin-4-ylchromen-4-one. It is a known DNA-PK inhibitor and has the following structure:

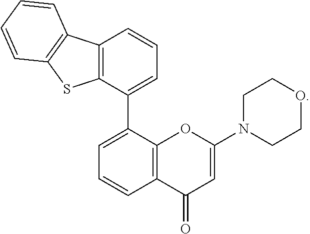 [SMILES: O=c1cc(N2CCOCC2)oc2c(-c3cccc4c3sc3ccccc34)cccc12]

LY3023414 is 8-[5-(2-hydroxypropan-2-yl)pyridin-3-yl]-1-[(2S)-2-methoxypropyl]-3-methylimidazo[4,5-c]quinolin-2-one. It is a known mTOR/DNA-PK and has the following structure:

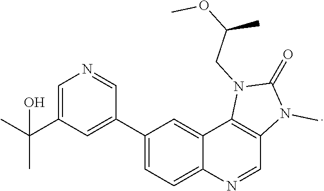 [SMILES: CO[C@@H](C)Cn1c(=O)n(C)c2cnc3ccc(-c4cncc(C(C)(C)O)c4)cc3c21]

### Protocols:

### MTT Assay Protocol:

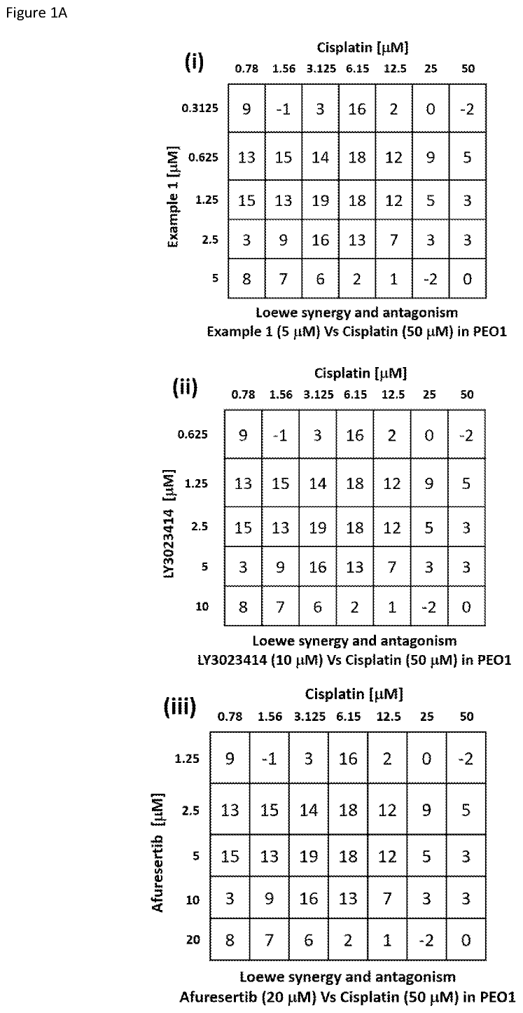

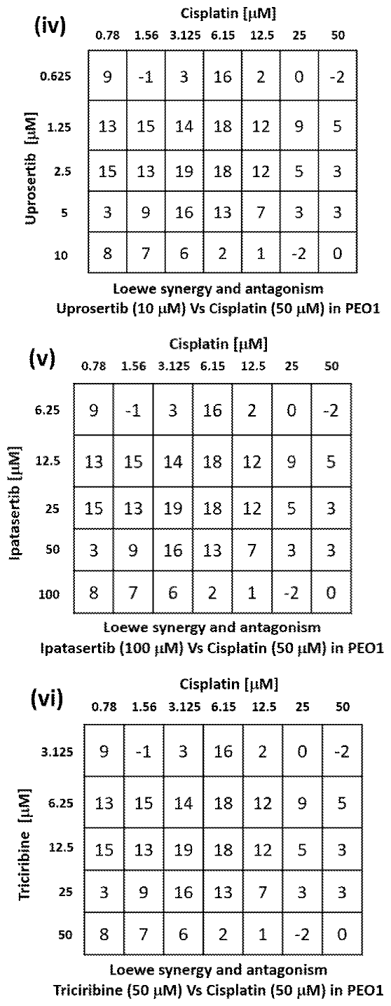

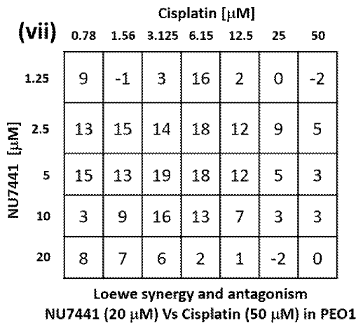

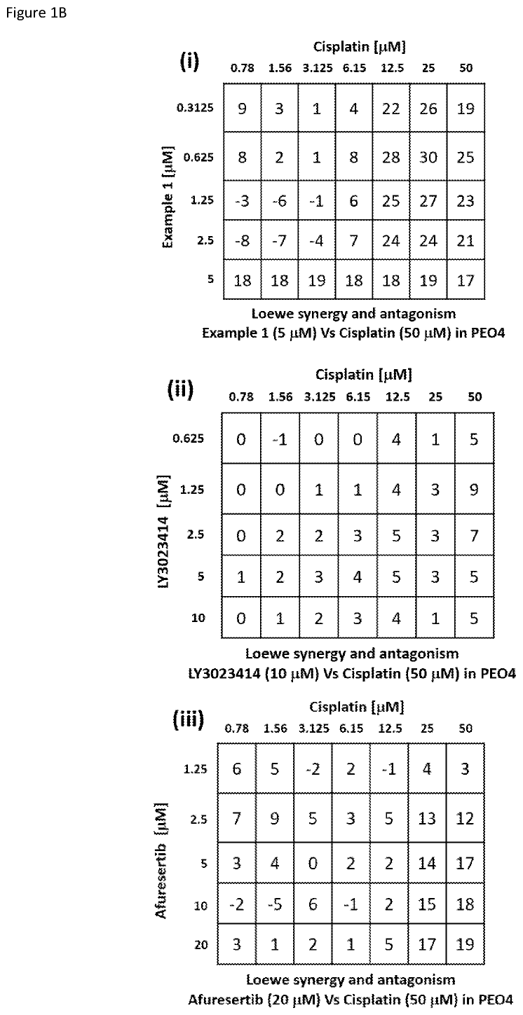

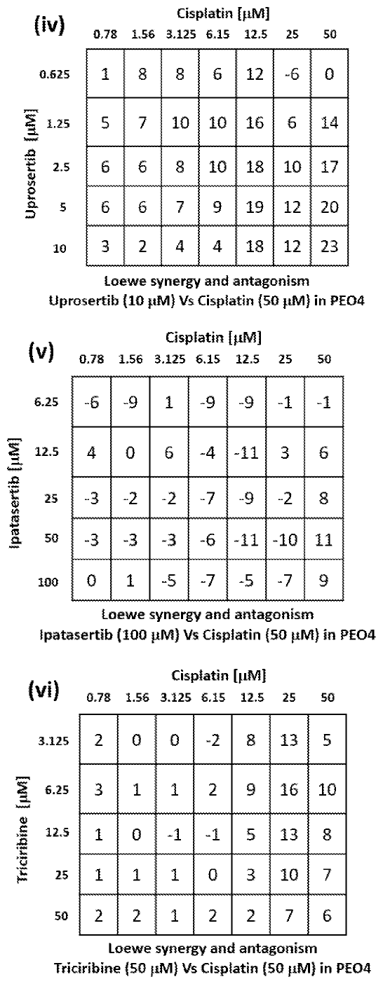

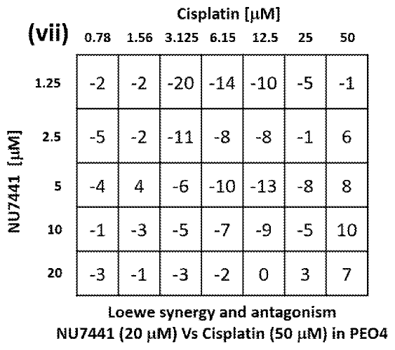

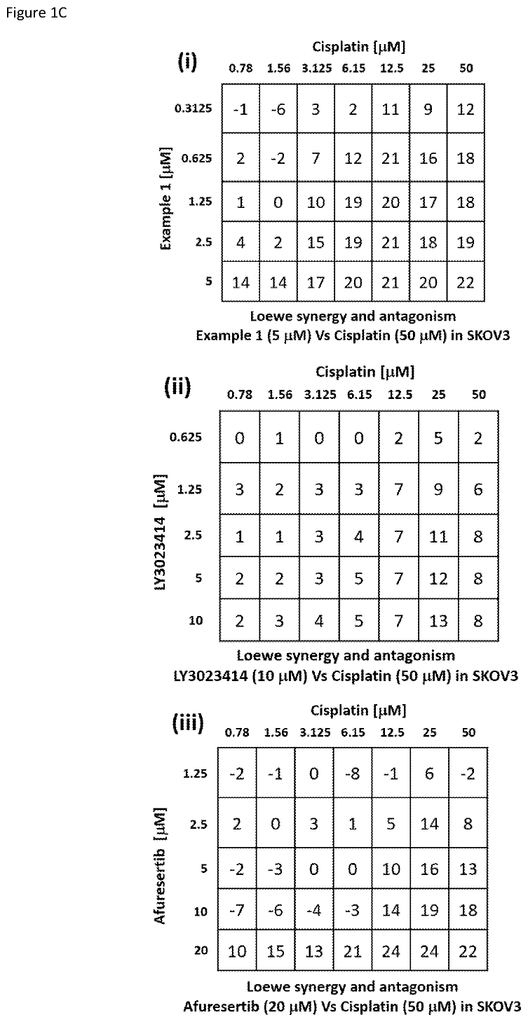

Cell viability was determined for different assays (as described in FIGS. 1 and 2, Table 5) in optically clear 96 well plates using the standard 3-(4, 5-dimethylthiazol-2-yl)-2, 5-diphenyl tetrazolium bromide (MTT) assay, which is a colorimetric method for determining the number of metabolically active cells in cytotoxicity assays. MTT is bioreduced by mitochondrial dehydrogenase enzymes into a coloured solid formazan product. MTT reagent (Sigma, UK) was initially prepared by dissolving in PBS at a concentration of 3 mg/ml. Following relevant drug treatments at different time points and cell lines (described in results section and FIGS. 1 and 2), MTT reagent was added to each experimental well at a final concentration of 0.5 mg/ml. After incubation for 3 h at 37° C. 5% CO2, an equal volume of stop solution (10% SDS in 0.01% HCl) was added and formed formazan crystals were allowed to dissolve shaking overnight. The following day, the optical density was read at 570 nm and analysis performed as per each assay type.

### Caspase Assay Protocol:

The caspase 3/7 activity of cells following treatment with inhibitor compounds or vehicle (DMSO) was determined using the Caspase-Glo® 3/7 Assay (Catalogue number G8093, Promega, US) according to manufacturer's instructions. Briefly, 10,000 cells per well for PEO1 and PEO4 cell lines or 5000 cells per well for SKOV3 cell line were grown in 96 well plates in a final volume of 50 μl and following relevant drug treatment of cells (example compound±cisplatin, comparative example compound±cisplatin, or vehicle±cisplatin) for 24 hours, an equal volume of Caspase-Glo® Reagent containing a luminogenic substrate for Caspase 3 and Caspase 7 was added i.e. if cells cultured in 50 μl of media, 50 μl of caspase reagent was added. After 1 h incubation at room temperature in the dark, the luminescence of each sample was measured in a plate-reading luminometer (LUMIstar OPTIMA, BMG Labtech, Germany). Caspase 3/7 activity data was normalised to corresponding viability data from MTT and analysis was performed according to each experimental set-up.

### Collection of Protein Lysates for Reverse Phase Protein Array Proteomic Analysis.

SKOV3 cells were seeded into 6 well plates at 200,000 cells per well. Cells were treated with inhibitor compounds listed in results section at 6.67 μM, 1 h later cisplatin at 25 μM or media control was added, and cells were incubated for two different time points (2 and 8 hours) before lysate collection. Protein lysates were collected according to the standard protocol for cell lysates from RPPA Functional Proteomics Facility, MD Anderson. Protein concentration was adjusted to 1 mg/ml and lysates were submitted to the facility for RPPA analysis.

### Results:

### Isobologram Experiments to Determine Synergy or Antagonism Between Example Compounds, Comparator Compounds and Platinum Chemotherapy:

Firstly, IC₅₀ experiments were carried out to determine optimal concentration ranges for synergy/antagonism assays for Example 1 and the comparative examples (LY3023414, Afuresertib, Uprosertib, Ipatasertib, triciribine and NU7441, all obtained from Selleck Chem). All cell lines (PEO1, PEO4 and SKOV3) were seeded out 24 hours prior to treatment into 96 well plates at 10,000 cells per well in triplicate for each inhibitor compound. Cells were treated with each inhibitor at uniformly reducing concentrations (serial 1:2 dilution), against a media control. Following 72 hours incubation, cell viability was assessed using the MTT assay (n=3). IC₅₀ values for each compound was determined using GraphPad Prism software, non-linear regression of log (inhibitor) versus response (three parameters). All isobologram assays used the standard MTT protocol as described above for assay readout. Cell lines were seeded out 24 hours prior to treatment in to 96 well, clear-bottomed plates at 10,000 cells per well for PE01 and PEO4 lines, and 5000 per well for SKOV3. Cells were treated with each compound (Example 1, LY3023414, Afuresertib, Uprosertib, Ipatasertib, triciribine or NU7441, FIGS. 1A-C)) at concentration ranges devised from IC₅₀ assays, at uniformly reducing concentrations (serial 1:2 dilutions), against a media control. Following 1 hour incubation, cells were treated with cisplatin at uniformly reducing concentrations (serial 1:2 dilutions starting at 50 μM cisplatin). Plates were incubated for 72 hours at 37° C. 5% CO₂. Following 72 h incubation, cell viability was assessed using MTT assays as described above. Analysis was performed using Combenefit Analysis LOEWE programme of synergy and antagonism, from Cambridge University (https://www.cruk.cam.ac.uk/research-groups/jodrell-group/combenefit). An increasing positive number in the isobologram analysis indicates increasing synergy between inhibitor compound and cisplatin. A decreasing negative number in the isobologram analysis indicates increasing antagonism between inhibitor compound and cisplatin.

FIGS. 1A, 1B and 1C show the isobologram analysis for the PEO1, PEO4 and SKOV3 cell lines respectively. As can be seen from FIGS. 1A to 1C, Example 1, which is a dual AKT/DNAPK inhibitor of the present invention, demonstrates superior synergy in combination with cisplatin compared to the comparative example compounds, which are a panel of currently commercially available AKT (Afuresertib, Uprosertib, Ipatasertib, triciribine), DNA-PK (NU7441) or mTOR/DNAPK (LY3023414) inhibitors. In particular strong synergy (increasing positive number) was observed for the Example 1 compound in combination with cisplatin in both platinum resistant cell lines (PEO4 and SKOV3) at a number of different concentrations (See FIGS. 1B and 1C), including very low concentrations of Example 1. In comparison to Example 1, weaker synergy or antagonism was observed for the combinations of the currently commercially available AKT, DNA-PK or mTOR/DNAPK inhibitors and cisplatin across all three cell line models.

Phenotypic Apoptosis Assays to Demonstrate Re-Sensitisation of Platinum Resistant Cells to Cisplatin when Treated with Example 1 AKT/DNAPK Inhibitor Compound

Phenotypic apoptosis assays were performed to determine the level of caspase 3/7 dependent apoptotic induction of each inhibitor compound in the presence or absence of cisplatin (cddp). PEO4 and SKOV3 cells were seeded out 24 hours prior to drug treatments. Cells were treated with different inhibitor concentrations; Example 1 at 2 μM concentration and 5 commercially available inhibitors (all obtained from Selleck Chem) that inhibit mTOR/DNAPK (LY3023414, 10 μM), AKT (Afuresertib, 10 μM, Uprosertib 10 μM, triciribine 20 μM, Ipatasertib 20 μM), or DNA-PK (NU7441 10 μM), or DMSO vehicle control, and incubated for 1 hr at 37° C. 5% CO₂. Following incubation, 25 μM cisplatin or media control was added to cells and incubated for 24 hours. Duplicate plates were set up for each experiment for caspase and MTT assays. Caspase and MTT assays were carried out according to the protocols described above.

The caspase 3/7 assay results were normalised to the standard MTT viability assay results of each corresponding well and the results are shown in FIGS. 2A (Example 1 and various comparative example compounds in SKOV3 cell line) and 2B (Example 1 and various comparative example compounds in PEO4 cell line). The results in FIGS. 2A and 2B are depicted as a fold change in apoptosis compared to the DMSO vehicle control. Data shown in FIGS. 2A and 2B are n=3±s.e.m. Statistical significance was calculated using student t-test using GraphPad Prism software ** p<0.01, * p<0.05.

As can be seen from FIG. 2A and FIG. 2B, statistically significant increases in apoptosis (re-sensitisation) was observed for the dual AKT/DNAPK inhibitor Example 1 (p<0.01) in combination with cisplatin, compared to cisplatin alone in both platinum resistant cell lines tested. The comparative example compounds tested in combination with cisplatin (Afuresertib, Uprosertib, Ipatasertib, triciribine, NU7441, or LY3023414) also showed re-sensitisation of platinum resistant cells to cisplatin in both platinum resistant cell lines, however at 5 or 10 times the dosage of Example 1. Therefore, Example 1 in combination with cisplatin, was able to re-sensitise platinum resistant cells to cisplatin at 10% or 20% of the inhibitor concentration required to elicit a similar apoptotic response for the other AKT, DNAPK or DNAPK/mTOR inhibitor compounds, Afuresertib, Uprosertib, Ipatasertib, triciribine, NU7441, or LY3023414. This demonstrates that the dual AKT/DNA-PK inhibitor compounds of the invention are especially effective at re-sensitising platinum resistant cells to cisplatin.

### Relative Re-Sensitisation Ratio of Apoptosis Induction (Caspase 3/7) of Inhibitor Compounds Relative to Triciribine Phosphate.

PEO4 cells, seeded out 24 h prior at 10,000 cells per well, were treated with a compound of the invention as listed in table 5 at 1, 5, 10 and 20 μM concentrations, or triciribine at 25 μM, for 1 hour. Cisplatin at a final concentration of 25 μM or media control was added and incubated for 24 h at 37° C. 5% CO₂. Apoptosis (caspase 3/7) and viability (MTT) assays were carried out as described for each assay protocols and the phenotypic apoptosis assays above. Relative apoptosis induction was inferred from caspase 3/7 assay values normalised to the standard MTT viability assay values of each corresponding well. A relative re-sensitisation ratio of relative apoptosis induction of each compound (at 20 μM concentration) relative to apoptosis induction of triciribine was calculated for each compound. The results in Table 5 show that compounds of the invention had an equivalent or higher re-sensitisation ratio than triciribine, in combination with cisplatin treatment.

TABLE 5

| | Relative re-sensitisation ratio of |
|---|---|
| Example | apoptosis induction (caspase 3/7) |
| No. | relative to triciribine phosphate |
| 1 | 3.0 |
| 2 | 2.9 |
| 3a | 2.7 |
| 4 | 2.6 |
| 5 | 2.5 |
| 6 | 2.3 |
| 7 | 2.3 |
| 8 | 1.73 |
| 3 | 1.7 |
| 9 | 1.5 |
| 10 | 1.5 |
| 3b | 1.4 |
| 11 | 1.3 |
| 12a | 1.2 |
| 13 | 1.2 |
| 14 | 1.2 |
| 15 | 1.1 |
| 12b | 1.0 |
| Triciribine | 1.0 |
| phosphate | |

### Proteomic Analysis of Example Compound Inhibition in SKOV3 Cells

Reverse Phase Protein Arrays were carried out on protein lysates collected following treatment of SKOV3 cells with Example 1, 2, 12a or 12b of the present invention (active compounds); or one of the following comparative example compounds: GSK-3g (GSK-690693) or Roche comp-4 (Breitenlechner C B, Journal of Medicinal Chemistry, 2004, 47, issue 6, pages 1375-1390), that were demonstrated to be inactive in phenotypic in vitro apoptosis platinum re-sensitisation assays in SKOV3 cells at the equivalent concentration of inhibitor at 6.67 μM and cisplatin at 25 μM (inactive compounds); or DMSO (control) plus/minus cisplatin. RPPA assays were performed in the RPPA Functional Proteomics Facility, MD Anderson, USA as per standard protocol (Tibes R. et al, Mol Cancer Ther (2006), Vol 5(10), Pages 2512-21, https://www.mdanderson.org/research/research-resources/core-facilities/functional-proteomics-rppa-core/technical-information.html).

GSK-3g (GSK-690693) and Roche comp-4 are known AKT inhibitors, as described above, and were synthesised as described above.

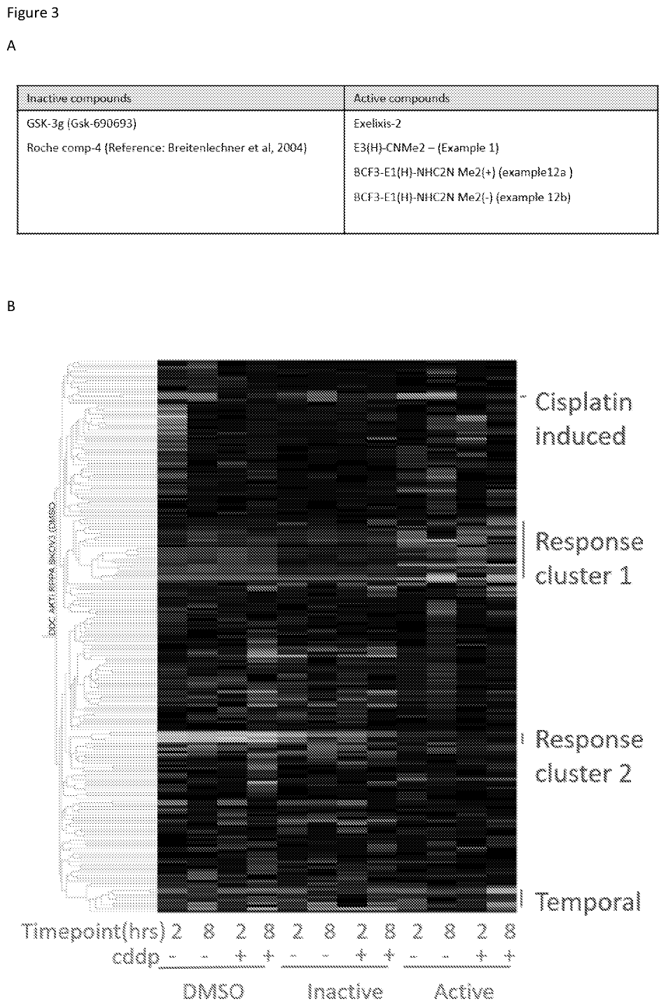

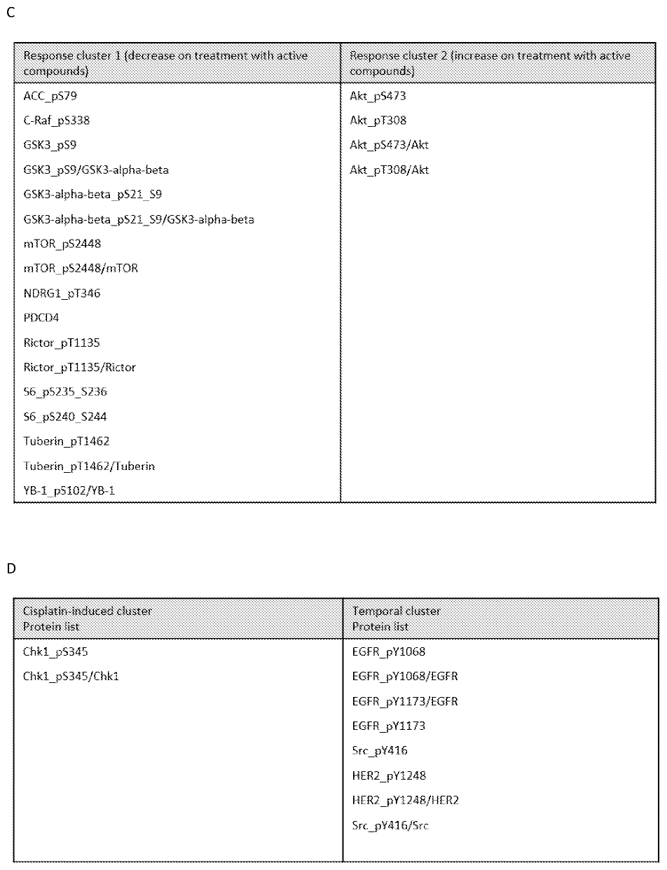

In this assay, increased phosphorylation of AKT at Serine473 and Threonine308 is observed upon treatment with active compounds. The compounds tested were grouped as either inactive or active plus/minus cisplatin due to phenotypic in vitro apoptosis platinum re-sensitisation assays (FIG. 3A) and a heatmap demonstrating signalling changes at different response clusters (1 and 2) or signalling changes induced due to cisplatin or temporal changes was generated (FIG. 3B). FIG. 3C lists the proteins that either decreased upon active inhibitor treatments (Response cluster 1—left panel) or increased following active inhibitor treatments (Response cluster 2—right panel) in combination with cisplatin. Increased AKT phosphorylation at Serine473 (AKT pS473) and at Threonine308 (AKT pT308) in response cluster 2 and decreased phosphorylation of proteins downstream of AKT signalling e.g. S6 at pS235-S236, pS240-S244 observed in response cluster 1, demonstrates that the compounds of the invention tested targeted the AKT pathway.

### SUMMARY

The results of the examples herein show that the dual AKT/DNA-PK inhibitor compounds of the invention work synergistically with cisplatin, particularly in the platinum resistant cell lines tested, and restore apoptotic function and response to platinum at much lower doses than the comparative example compounds that are inhibitors of AKT, DNA-PK or mTOR/DNA-PK. Proteomic analysis demonstrated increased phosphorylation of AKT at S473 and T308 by the compounds of the invention in combination with cisplatin treatment, and downstream targeting of AKT pathway molecules.

1. A compound of formula (I) for use in the treatment of a platinum resistant cancer, 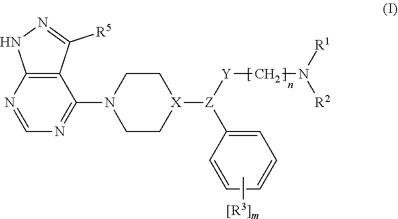
wherein
X is CH, and Z is CR⁴ or N; or X is N, and Z is CR⁴; or X—Z is C═C;
R¹ and R² are independently selected from H, C₁₋₄alkyl, C₃₋₆cycloalkyl and 5-6 membered heterocyclyl, or R¹ and R² together with the nitrogen to which that are attached form a 4-10 membered heterocyclyl ring;
each R³ is independently selected from methyl, halogen and CF₃;
m is 0, 1, 2 or 3;
R⁴ is H or OH; and
when Z is N, or X—Z is C═C:
Y is selected from bond, —C(O)— and —SO₂NH—, and R⁵ is selected from methyl, halogen and CF₃; or
Y is selected from —SO₂—, and R⁵ is selected from methyl and halogen; or
Y is —C(O)NR⁶—, and R⁵ is halogen;
when Z is CR⁴:
Y is selected from bond, —C(O)—, —SO₂NH—, and —NHSO₂—, and R⁵ is selected from methyl, halogen and CF₃; or
Y is selected from —SO₂—, and R⁵ is selected from methyl and halogen; or
Y is —C(O)NR⁶—, and R⁵ is halogen;
Y is —NR⁶—, and R⁵ is selected from methyl, halogen and CF₃; or
Y is —O—, and R⁵ is selected from methyl and halogen; and
when Y is selected from bond, —SO₂NH—, —O—, —NR⁶— and —C(O)NR⁶—, n is 1, 2, 3 or 4;
when Y is selected from —NHSO₂—, —SO₂— and —C(O)—, n is 0, 1, 2, 3 or 4; and
R⁶ is selected from H and C₁₋₄alkyl;
or a salt thereof.

2. The compound for use as claimed in claim 1, wherein Y is selected from bond, —C(O)—, —O— and —NR⁶—, or
wherein R¹ and R² are each C₁₋₄alkyl; or
wherein m is 1 and R³ is chloro, for example m is 1 and R³ is para-chloro; or
wherein R⁴ is H; or
wherein R⁵ is bromo; or
wherein n is 1 (for example, wherein n is 1 and Y is bond).

3.-7. (canceled)

8. The compound for use according to claim 1, wherein the compound is selected from:
2-(4-(3-bromo-1H-pyrazolo[3,4-d]pyrimidin-4-yl)piperazin-1-yl)-2-(4-chlorophenyl)-N,N-dimethylethan-1-amine;
2-((1-(3-bromo-1H-pyrazolo[3,4-d]pyrimidin-4-yl)piperidin-4-yl)(4-chlorophenyl)methoxy)-N,N-dimethylethan-1-amine;
2-(4-(3-bromo-1H-pyrazolo[3,4-d]pyrimidin-4-yl)piperazin-1-yl)-2-(4-chlorophenyl)-N-(2-(dimethylamino)ethyl)acetamide;
(−)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
(+)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
2-(4-(3-bromo-1H-pyrazolo[3,4-d]pyrimidin-4-yl)piperazin-1-yl)-2-(4-chlorophenyl)-N,N-dimethylacetamide;
2-((4-chlorophenyl)(1-(3-methyl-1H-pyrazolo[3,4-d]pyrimidin-4-yl)piperidin-4-yl)methoxy)-N,N-dimethylethan-1-amine;
N-(1-(3-bromo-1H-pyrazolo[3,4-d]pyrimidin-4-yl)piperidin-4-yl)-N-(4-chlorophenyl)-2-(diethylamino)ethane-1-sulfonamide; and
2-(1-(3-bromo-1H-pyrazolo[3,4-d]pyrimidin-4-yl)piperidin-4-yl)-2-(4-chlorophenyl)-N,N-dimethylethan-1-amine;
N¹-((1-(3-bromo-1H-pyrazolo[3,4-d]pyrimidin-4-yl)piperidin-4-yl)(4-chlorophenyl)methyl)-N²,N²-dimethylethane-1,2-diamine;
2-(4-chlorophenyl)-N,N-dimethyl-2-(1-(3-(trifluoromethyl)-1H-pyrazolo[3,4-d]pyrimidin-4-yl)piperidin-4-yl)ethan-1-amine;
N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(+)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(−)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
2-(4-chlorophenyl)-N,N-dimethyl-2-(4-(3-(trifluoromethyl)-1H-pyrazolo[3,4-d]pyrimidin-4-yl)piperazin-1-yl)ethan-1-amine;
1-(1-(3-bromo-1H-pyrazolo[3,4-d]pyrimidin-4-yl)piperidin-4-yl)-1-(4-chlorophenyl)-5-(dimethylamino)pentan-1-ol;
4-(4-chlorophenyl)-N,N-dimethyl-4-(1-(3-(trifluoromethyl)-1H-pyrazolo[3,4-d]pyrimidin-4-yl)piperidin-4-ylidene)butan-1-amine;
1-(1-(3-bromo-1H-pyrazolo[3,4-d]pyrimidin-4-yl)piperidin-4-yl)-1-(4-chlorophenyl)-4-(dimethylamino)butan-1-ol; and
3-((1-(3-bromo-1H-pyrazolo[3,4-d]pyrimidin-4-yl)piperidin-4-yl)(4-chlorophenyl)methoxy)-N,N-dimethylpropan-1-amine.

9. The compound for use according to claim 1, wherein the cancer is ovarian cancer, for example high-grade serous ovarian cancer; or
wherein the compound is administered in combination with a platinum containing drug; or
wherein the compound is administered in combination with a further therapeutic agent.

10.-11. (canceled)

12. A method for the treatment of platinum containing drug resistant cancer, comprising administering a compound according to claim 1 to a subject in need thereof.

13. The compound for use according to claim 1 and a platinum containing drug for use in a combined treatment of cancer, whereby the compound and the platinum containing drug are administered simultaneously or separately; or
wherein the cancer is resistant to the platinum containing drug.

14. (canceled)

15. The compound and platinum containing drug for use according to claim 13 wherein the cancer is ovarian cancer (for example high-grade serous ovarian cancer); or
wherein the platinum containing drug is cisplatin or carboplatin; or
wherein the compound and platinum containing drug are administered in combination with a further therapeutic agent.

16.-17. (canceled)

18. The compound for use according to claim 1, wherein the platinum resistant cancer is a cancer that has progressed while receiving chemotherapeutic treatment using a platinum containing drug, or a cancer that has recurred within 18 months (for example within 12 months or within 6 months) of completion of treatment using a platinum containing drug.

19. A method for the treatment of a cancer, comprising administering a compound according to claim 1 and a platinum containing drug to a patient subject in need thereof in a combined treatment.

20. A pharmaceutical composition comprising a compound according to claim 1, a pharmaceutically suitable carrier and a platinum containing drug.

21. A compound of formula (Ia): 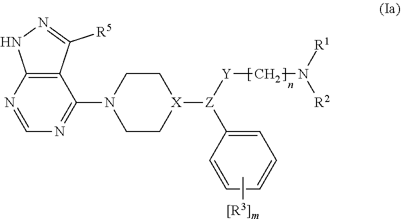
wherein
X is CH, and Z is CR⁴ or N; or X is N, and Z is CR⁴; or X—Z is C═C;
R¹ and R² are independently selected from H, C₁₋₄alkyl, C₃₋₆cycloalkyl and 5-6 membered heterocyclyl, or R¹ and R² together with the nitrogen to which that are attached form a 4-10 membered heterocyclyl ring;
each R³ is independently selected from methyl, halogen and CF₃;
m is 0, 1, 2 or 3;
R⁴ is H or OH; and
when Z is N, or X—Z is C═C:
Y is selected from bond, —C(O)— and —SO₂NH—, and R⁵ is selected from methyl, halogen and CF₃; or
Y is selected from —SO₂—, and R⁵ is selected from methyl and halogen; or
Y is —C(O)NR⁶—, and R⁵ is halogen;
when Z is CR⁴:
Y is selected from bond, —C(O)—, —SO₂NH—, and —NHSO₂—, and R⁵ is selected from methyl, halogen and CF₃; or
Y is selected from —SO₂—, and R⁵ is selected from methyl and halogen; or
Y is —C(O)NR⁶—, and R⁵ is halogen;
Y is —NR⁶—, and R⁵ is selected from methyl, halogen and CF₃; or
Y is —O—, and R⁵ is selected from methyl and halogen; and
when Y is selected from bond, —SO₂NH—, —O—, —NR⁶— and —C(O)NR⁶—, n is 1, 2, 3 or 4;
when Y is selected from —NHSO₂—, —SO₂— and —C(O)—, n is 0, 1, 2, 3 or 4; and
R⁶ is selected from H and C₁₋₄alkyl;
or a salt thereof, with the proviso that compound is not:
2-{[[1-(3-bromo-1H-pyrazolo[3,4-d]pyrimidin-4-yl)piperidin-4-yl](4-chlorophenyl)methyl]oxy}-N,N-dimethylethanamine
3-{[[1-(3-bromo-1H-pyrazolo[3,4-d]pyrimidin-4-yl)piperidin-4-yl](4-chlorophenyl)methyl]oxy}-N,N-dimethylpropan-1-amine
N′-[[1-(3-bromo-1H-pyrazolo[3,4-d]pyrimidin-4-yl)piperidin-4-yl](4-chlorophenyl)methyl]-N,N-diethylethane-1,2-diamine
2-[4-(3-bromo-1H-pyrazolo[3,4-d]pyrimidin-4-yl)piperazin-1-yl]-2-(4-chlorophenyl)-N-[2-(dimethylamino)ethyl]acetamide
N-[1-(3-bromo-1H-pyrazolo[3,4-d]pyrimidin-4-yl)piperidin-4-yl]-N-(4-chlorophenyl)-N′,N′-diethylpropane-1,3-diamine
N-[1-(3-bromo-1H-pyrazolo[3,4-d]pyrimidin-4-yl)piperidin-4-yl]-N-(4-chlorophenyl)-N′-[2-(dimethylamino)ethyl]urea
N-[1-(3-bromo-1H-pyrazolo[3,4-d]pyrimidin-4-yl)piperidin-4-yl]-N-(4-chlorophenyl)-N′,N′-diethylethane-1,2-diamine
N-[[1-(3-bromo-1H-pyrazolo[3,4-d]pyrimidin-4-yl)piperidin-4-yl](4-chlorophenyl)methyl]-N′,N′-diethyl-N-methylethane-1,2-diamine
N-[1-(3-bromo-1H-pyrazolo[3,4-d]pyrimidin-4-yl)piperidin-4-yl]-N-(4-chlorophenyl)-N˜3˜,N˜3˜-diethyl-beta-alaninamide
2-{[[1-(3-bromo-1H-pyrazolo[3,4-d]pyrimidin-4-yl)piperidin-4-yl](4-fluorophenyl)methyl]oxy}-N,N-dimethylethanamine
N-[1-(3-bromo-1H-pyrazolo[3,4-d]pyrimidin-4-yl)piperidin-4-yl]-N-(4-chlorophenyl)-2-(diethylamino)ethanesulfonamide
2-{[(R)-[1-(3-bromo-1H-pyrazolo[3,4-d]pyrimidin-4-yl)piperidin-4-yl](4-chlorophenyl)methyl]oxy}-N,N-dimethylethanamine
N-(4-bromo-3-fluorophenyl)-N-[1-(3-bromo-1H-pyrazolo[3,4-d]pyrimidin-4-yl)piperidin-4-yl]-N′-[2-(dimethylamino)ethyl]urea
2-{[(S)-[1-(3-bromo-1H-pyrazolo[3,4-d]pyrimidin-4-yl)piperidin-4-yl](4-chlorophenyl)methyl]oxy}-N,N-dimethylethanamine
3-bromo-4-(4-{(R)-(4-chlorophenyl)[(2-pyrrolidin-1-ylethyl)oxy]methyl}piperidin-1-yl)-1H-pyrazolo[3,4-d]pyrimidine
1-[1-(3-bromo-1H-pyrazolo[3,4-d]pyrimidin-4-yl)piperidin-4-yl]-1-(4-chlorophenyl)-4-(dimethylamino)butan-1-ol
2-{[(R)-[1-(3-bromo-1H-pyrazolo[3,4-d]pyrimidin-4-yl)piperidin-4-yl](4-chloro-3-fluorophenyl)methyl]oxy}-N,N-dimethylethanamine
3-bromo-4-(4-{(R)-(4-chlorophenyl)[(2-piperidin-1-ylethyl)oxy]methyl}piperidin-1-yl)-1H-pyrazolo[3,4-d]pyrimidine
4-[1-(3-bromo-1H-pyrazolo[3,4-d]pyrimidin-4-yl)piperidin-4-yl]-4-(4-chlorophenyl)-N,N-dimethylbutan-1-amine
3-bromo-4-(4-{(R)-(4-chlorophenyl)[(2-morpholin-4-ylethyl)oxy]methyl}piperidin-1-yl)-1H-pyrazolo[3,4-d]pyrimidine
3-bromo-4-(4-{(R)-(4-chloro-3-fluorophenyl)[(2-pyrrolidin-1-ylethyl)oxy]methyl}piperidin-1-yl)-1H-pyrazolo[3,4-d]pyrimidine
2-{[(R)-[1-(3-bromo-1H-pyrazolo[3,4-d]pyrimidin-4-yl)piperidin-4-yl](4-chlorophenyl)methyl]oxy}-N,N-diethylethanamine.

22. A compound according to claim 21, wherein the compound is a compound of formula (II) 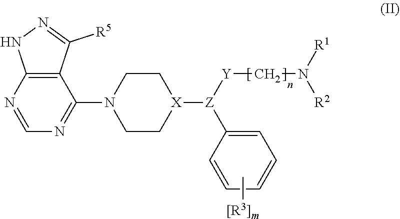
wherein
X is CH, and Z is CR⁴ or N; or X is N, and Z is CR⁴; or X—Z is C═C;
R¹ and R² are independently selected from H, C₁₋₄alkyl, C₃₋₆cycloalkyl and 5-6 membered heterocyclyl, or R¹ and R² together with the nitrogen to which that are attached form a 4 to 10 membered heterocyclyl ring;
each R³ is independently selected from methyl, halogen and CF₃;
m is 0, 1, 2 or 3;
R⁴ is H or OH;
when Z is N, or X—Z is C═C:
Y is selected from bond, —C(O)— and —SO₂NH—, and R⁵ is selected from methyl, halogen and CF₃; or
Y is —SO₂—, and R⁵ is selected from methyl and halogen; or
Y is —C(O)NR⁶—, and R⁵ is halogen;
when Z is CR⁴:
Y is selected from bond, —C(O)—, —SO₂NH— and —NHSO₂—, and R⁵ is selected from methyl, halogen and CF₃; or
Y is —SO₂—, and R⁵ is selected from methyl and halogen; or
Y is —C(O)NR⁶—, and R⁵ is halogen;
Y is —NR⁶—, and R⁵ is selected from methyl, halogen and CF₃; or
Y is —O—, and R⁵ is methyl; and
when Y is selected from —C(O)NR⁶— and —NR⁶—, n is 1, 3 or 4;
when Y is —SO₂NH— or —O—, n is 1, 2, 3 or 4;
when Z is CR⁴ or X—Z is C═C, and Y is bond, n is 1, 2 or 4;
when Z is N, and Y is bond, n is 1 or 4;
when Z is CR⁴ or X—Z is C═C, and Y is selected from —NHSO₂—, —SO₂— and —C(O)—, n is 0, 1, 2, 3 or 4;
when Z is N, and Y is selected from —SO₂— and —C(O)—, n is 0, 1, 3 or 4; and
R⁶ is selected from H and C₁₋₄alkyl;
or a salt thereof.

23. The compound according to claim 21, wherein Z is CR⁴; or
wherein Y is selected from bond, —C(O)—, —O— and —NR⁶—; or
wherein R¹ and R² are each C₁₋₄alkyl; or
wherein m is 1 and R³ is chloro, for example m is 1 and R³ is para-chloro; or
wherein Z is CR⁴, Y is —O—, and R⁵ is methyl; or
wherein Y is selected from bond, —C(O)— and —NR⁶—, and R⁵ is bromo;
wherein X is CH, Z is CR₄ and Y is bond; or
wherein n is 1.

24.-30. (canceled)

31. The compound according to claim 21 wherein the compound is selected from:
2-[4-(3-bromo-1H-pyrazolo[3,4-d]pyrimidin-4-yl)piperazin-1-yl]-2-(4-chlorophenyl)-N,N-dimethyl-ethanamine;
2-(4-(3-bromo-1H-pyrazolo[3,4-d]pyrimidin-4-yl)piperazin-1-yl)-2-(4-chlorophenyl)-N,N-dimethylacetamide;
2-((4-chlorophenyl)(1-(3-methyl-1H-pyrazolo[3,4-d]pyrimidin-4-yl)piperidin-4-yl)methoxy)-N,N-dimethylethan-1-amine
2-(1-(3-bromo-1H-pyrazolo[3,4-d]pyrimidin-4-yl)piperidin-4-yl)-2-(4-chlorophenyl)-N,N-dimethylethan-1-amine;
(−)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
(+)-2-(4-(3-bromo-1H-pyrazolo[3,4-d]pyrimidin-4-yl)piperazin-1-yl)-2-(4-chlorophenyl)-N-(2-(dimethylamino)ethyl)acetamide;
N¹-((1-(3-bromo-1H-pyrazolo[3,4-d]pyrimidin-4-yl)piperidin-4-yl)(4-chlorophenyl)methyl)-N²,N²-dimethylethane-1,2-diamine;
2-(4-chlorophenyl)-N,N-dimethyl-2-(1-(3-(trifluoromethyl)-1H-pyrazolo[3,4-d]pyrimidin-4-yl)piperidin-4-yl)ethan-1-amine;
N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(+)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
(−)-N¹-((4-chlorophenyl)(1-(3-(trifluoromethyl)-1H-pyrazolo[3,4-d]pyrimidin-4-yl)piperidin-4-yl)methyl)-N²,N²-dimethylethane-1,2-diamine;
2-(4-chlorophenyl)-N,N-dimethyl-2-(4-(3-(trifluoromethyl)-1H-pyrazolo[3,4-d]pyrimidin-4-yl)piperazin-1-yl)ethan-1-amine;
1-(1-(3-bromo-1H-pyrazolo[3,4-d]pyrimidin-4-yl)piperidin-4-yl)-1-(4-chlorophenyl)-5-(dimethylamino)pentan-1-ol; and
4-(4-chlorophenyl)-N,N-dimethyl-4-(1-(3-(trifluoromethyl)-1H-pyrazolo[3,4-d]pyrimidin-4-yl)piperidin-4-ylidene)butan-1-amine.

32. A pharmaceutical composition comprising a compound according to claim 21, a pharmaceutically suitable carrier and optionally a further therapeutic agent (for example a platinum containing drug).

33. (canceled)

34. A compound according to claim 21, for use in the treatment of cancer.

35. The compound for use according to claim 34, wherein the platinum resistant cancer is ovarian cancer; and/or wherein the platinum resistant cancer is a cancer that has progressed while receiving chemotherapeutic treatment using a platinum containing drug, or a cancer that has recurred within 18 months of completion of treatment using a platinum containing drug.
